SEQUENCE LISTING (367 sequences)

SEQ ID NO: 1 - protein, 95 aa, Mus musculus
STTVTQSPASLSMAIGEKVTIRCITSTDIDDDMNWYQQKPGEPPKLLISEGNTLRPGVPSRFSSSGYGTDFVFTIENMLSEDVADYYCLQSDNLP

SEQ ID NO: 2 - protein, 95 aa, Mus musculus
ETTVTQSPASLSVATGEKVTIRCITSTDIDDDMNWYQQKPGEPPKLLISEGNTLRPGVPSRFSSSGYGTDFVFTIENTLSEDVADYYCLQSDNMP

SEQ ID NO: 3 - protein, 95 aa, Mus musculus
DIQMTQSPSSLSASLGGKVTITCKASQDINKYIAWYQHKPGKGPRLLIHYTSTLQPGIPSRFSGSGSGRDYSFSISNLEPEDIATYYCLQYDNLL

SEQ ID NO: 4 - protein, 103 aa, Mus musculus
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFV

SEQ ID NO: 5 - protein, 99 aa, Mus musculus
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDP

SEQ ID NO: 6 - protein, 95 aa, Mus musculus
DIQMTQSPASLAASVGETVTITCRASENIYYSLAWYQQKQGKSPQLLIYNANSLEDGVPSRFSGSGSGTQYSMKINSMQPEDTATYFCKQAYDVP

SEQ ID NO: 7 - protein, 95 aa, Mus musculus
DIQMTQSPASLSVSVGETVTITCRASENIYSNLAWYQQKQGKSPQLLVYAATNLADGVPSRFSGSGSGTQYSLKINSLQSEDFGSYYCQHFWGTP

SEQ ID NO: 8 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 9 - protein, 96 aa, Homo sapiens
EIVMTQSPATLSLSPGERATLSCRASQSVSSSYLSWYQQKPGQAPRLLIYGASTRATGIPARFSGSGSGTDFTLTISSLQPEDFAVYYCQQDYNLP

SEQ ID NO: 10 - protein, 95 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFP

SEQ ID NO: 11 - protein, 95 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 12 - protein, 97 aa, Homo sapiens
EIVMTQSPPTLSLSPGERVTLSCRASQSVSSSYLTWYQQKPGQAPRLLIYGASTRATSIPARFSGSGSGTDFTLTISSLQPEDFAVYYCQQDHNLPP

SEQ ID NO: 13 - protein, 95 aa, Homo sapiens
SYELTQPSSVSVSPGQTARITCSGDVLAKKYARWFQQKPGQAPVLVIYKDSERPSGIPERFSGSSSGTTVTLTISGAQVEDEADYYCYSAADNNL

SEQ ID NO: 14 - protein, 95 aa, Homo sapiens
SYELTQLPSVSVSPGQTARITCSGDVLGENYADWYQQKPGQAPELVIYEDSERYPGIPERFSGSTSGNTTTLTISRVLTEDEADYYCLSGDEDNP

SEQ ID NO: 15 - protein, 99 aa, Homo sapiens
QPVLTQSSSASASLGSSVKLTCTLSSGHSSYIIAWHQQQPGKAPRYLMKLEGSGSYNKGSGVPDRFSGSSSGADRYLTISNLQFEDEADYYCETWDSNT

SEQ ID NO: 16 - DNA, 67 bp, Homo sapiens
ctgcaggtctagtcaaagcctcgtatacagtgatggaaacacctacttgaattggtttcagcagagg

SEQ ID NO: 17 - DNA, 67 bp, Homo sapiens
ctcaccatcaatagcctggaagctgaagatgctgcaacgtattactgtcatcagagtagtagtttac

SEQ ID NO: 18 - DNA, 67 bp, Homo sapiens
gacttcactctcaccatcagcagactggagcctgaagattttgcagtgtattactgtcagcagtatg

SEQ ID NO: 19 - DNA, 67 bp, Homo sapiens
gatctgggacagatttcacctttaccatcagtagcctggaagctgaagatgctgcaacatattactg

SEQ ID NO: 20 - DNA, 67 bp, Homo sapiens
catctcctgcaagtctagtcagagcctcctgcatagtgatggaaagacctatttgtattggtacctg

SEQ ID NO: 21 - DNA, 67 bp, Homo sapiens
gatcaggcacagattttacactgaaaatcagcagagtggaggctgaggatgttggggtttattactg

SEQ ID NO: 22 - DNA, 67 bp, Homo sapiens
tctgcatctgtaggagacagagtcaccatcacttgccgggcgagtcagggcattagcaattatttag

SEQ ID NO: 23 - DNA, 67 bp, Homo sapiens
ctacttgagttggcttcagcagaggccaggccagcctccaagactcctaatttataagatttctaac

SEQ ID NO: 24 - DNA, 67 bp, Homo sapiens
ctctcacaatcagcagcctgcagcctgaagattttgcaacttattactgtctacagcataatagtta

SEQ ID NO: 25 - DNA, 67 bp, Homo sapiens
catctccttcaggtctagtcaaagcctcgtacacagtgatggaaacacctacttgagttggcttcag

SEQ ID NO: 26 - DNA, 67 bp, Homo sapiens
gagaagctgctattttcattattcaagaagctactactctcgttcctggaatcccacctcgattcag

SEQ ID NO: 27 - DNA, 67 bp, Homo sapiens
catcaactgcaagtccagccagagtgttttatacagctccaacaataagaactacttagcttggtac

SEQ ID NO: 28 - DNA, 67 bp, Homo sapiens
gtaggagacagagtcaccatcacttgtcgggcgagtcagggcattagcaattatttagcctggtttc

SEQ ID NO: 29 - DNA, 67 bp, Homo sapiens
ctcaccatcagcagcctgcagcctgaagattttgcagtttattactgtcagcaggatcataacttac

SEQ ID NO: 30 - DNA, 67 bp, Homo sapiens
ctctcaccatcagcagcctgcagcctgaagattttgcaacttattactgtctacaagattacaatta

SEQ ID NO: 31 - DNA, 67 bp, Homo sapiens
tggtatcagcagaaaccagggaaagcccctaagctcctgatctatgatgcctccagtttggaaagtg

SEQ ID NO: 32 - DNA, 67 bp, Homo sapiens
ctctcaccatcagcagcctgcagcctgaagattttgcaacttattactgccaacagtataatagtta

SEQ ID NO: 33 - DNA, 67 bp, Homo sapiens
ctctcaccatcagcagcctgcagtctgaagattttgcagtttattactgtcagcagtataataactg

SEQ ID NO: 34 - DNA, 67 bp, Homo sapiens
tggtatcagcagaaaccagggaaagctcctaagctcctgatctatgatgcctccagtttggaaagtg

SEQ ID NO: 35 - DNA, 67 bp, Homo sapiens
catcttctgtgtctgcatctgtaggagacagagtcaccatcacttgtcgggcgagtcagggtattag

SEQ ID NO: 36 - DNA, 67 bp, Homo sapiens
ttcactctcaccatcagcagcctagagcctgaagattttgcagtttattactgtcagcagcgtagca

SEQ ID NO: 37 - DNA, 67 bp, Homo sapiens
gtaggagacagagtcagtatcatttgctgggcaagtgagggcattagcagtaatttagcctggtatc

SEQ ID NO: 38 - DNA, 67 bp, Homo sapiens
ctctcaccatcagcagcctgcagcctgaagattttgcaacttattactgtcaacagtattatagtac

SEQ ID NO: 39 - DNA, 67 bp, Homo sapiens
catctacaggagacagagtcaccatcagttgtcggatgagtcagggcattagcagttatttagcctg

SEQ ID NO: 40 - DNA, 67 bp, Homo sapiens
ctcaccatcagcagcctgcagcctgaagattttgcagtttattactgtcagcaggattataacttac

SEQ ID NO: 41 - DNA, 67 bp, Homo sapiens
gacagatttcactctcaccatcagcagcctgcagcctgaagattttgcaacttactattgtcaacag

SEQ ID NO: 42 - DNA, 67 bp, Homo sapiens
ctctcacaatcagcagcctgcagcctgaagattttgcaacttattactgtcaacagcttaatagtta

SEQ ID NO: 43 - DNA, 67 bp, Homo sapiens
catctacaggagacagagtcaccatcacttgtcgggcgagtcagggtattagcagttatttagcctg

SEQ ID NO: 44 - DNA, 67 bp, Homo sapiens
gtctagtcagagcctcttggatagtgatgatggaaacacctatttggactggtacctgcagaagcca

SEQ ID NO: 45 - DNA, 67 bp, Homo sapiens
gagcattagcagctatttaaattggtatcagcagaaaccagggaaagcccctaagctcctgatctat

SEQ ID NO: 46 - DNA, 67 bp, Homo sapiens
cagttatttaaattggtatcggcagaaaccagggaaagttcctaagctcctgatctatagtgcatcc

SEQ ID NO: 47 - DNA, 67 bp, Homo sapiens
gtaggagacagagtcaccatcacttgccaggcgagtcaggacattagcaactatttaaattggtatc

SEQ ID NO: 48 - DNA, 67 bp, Homo sapiens
gcagagggtcaccatctcctgttctggaagcagctccaacatcggaaataatgctgtaaactggtac

SEQ ID NO: 49 - DNA, 67 bp, Homo sapiens
gcagagggtcaccatctcttgttctggaagcagctccaacatcggaagtaatactgtaaactggtac

SEQ ID NO: 50 - DNA, 67 bp, Homo sapiens
gcagagggtcaccatctcttgttctggaagcagctccaacatcggaagtaattatgtatactggtac

SEQ ID NO: 51 - DNA, 67 bp, Homo sapiens
ctccctggccatcactggactccagtctgaggatgaggctgattattactgcaaagcatgggataac

SEQ ID NO: 52 - DNA, 67 bp, Homo sapiens
caaactcctcatttatgacaataataagcgaccctcagggattcctgaccgattctctggctccaag

SEQ ID NO: 53 - DNA, 67 bp, Homo sapiens
gtcagtcaccatctcctgcactggaaccagcagtgacgttggtggttataactatgtctcctggtac

SEQ ID NO: 54 - DNA, 67 bp, Homo sapiens
ccaccctcccaaactcctatcctacaggaataacaaccggccctcagggatctcagagagattctct

SEQ ID NO: 55 - DNA, 67 bp, Homo sapiens
ctgtgatctatgaggataaccaaagaccctctggggtccctgatcggttctctggctccatcgacag

SEQ ID NO: 56 - DNA, 67 bp, Homo sapiens
gtcagtcaccatctcctgcactggaaccagcagtgatgttggtggttataactatgtctcctggtac

SEQ ID NO: 57 - DNA, 67 bp, Homo sapiens
caaactcatgatttatgaggtcagtaatcggccctcaggggtttctaatcgcttctctggctccaag

SEQ ID NO: 58 - DNA, 67 bp, Homo sapiens
ctctgggctccaggctgaggacgaggctgattattactgcagcttatatacaagcagcagcactttc

SEQ ID NO: 59 - DNA, 68 bp, Homo sapiens
ctgacaatctctgggctccaggctgaggacgaggctgattattactgctgctcatatgcaggtagtag

SEQ ID NO: 60 - DNA, 67 bp, Homo sapiens
ctctgggctcaagtccgaggttgaggctaattatcactgcagcttatattcaagtagttacactttc

SEQ ID NO: 61 - DNA, 68 bp, Homo sapiens
gataaatatgcttgctggtatcagcagaagccaggccagtcccctgtgctggtcatctatcaagatag

SEQ ID NO: 62 - DNA, 67 bp, Homo sapiens
tagtcacattgaccatcagtggagtccaggcagaagacgaggctgactattactgtctatcagcaga

SEQ ID NO: 63 - DNA, 67 bp, Homo sapiens
cttgggacagacagtcaggatcacatgccaaggagacagcctcagaagctattatgcaagctggtac

SEQ ID NO: 64 - DNA, 67 bp, Homo sapiens
ctgagttggtgatatacgaagatagtgagcggtaccctggaatccctgaacgattctctgggtccac

SEQ ID NO: 65 - DNA, 68 bp, Homo sapiens
gacaacagtcacgttgaccatcagtggagtccaggcagaagatgaggctgactattactgtcaatcag

SEQ ID NO: 66 - DNA, 67 bp, Homo sapiens
ctgggacagacggccaggattacctgtgggggaaacaacattggaagtaaaaatgtgcactggtacc

SEQ ID NO: 67 - DNA, 67 bp, Homo sapiens
gttactatccaaactggttccagcagaaacctggacaagcacccagggcactgatttatagtacaag

SEQ ID NO: 68 - DNA, 67 bp, Homo sapiens
gacactgatttatgatacaagcaacaaacactcctggacacctgcccggttctcaggctccctcctt

SEQ ID NO: 69 - DNA, 67 bp, Homo sapiens
ctggagggacagtcacactcacttgtggcttgagctctggctcagtctctactagttactaccccag

SEQ ID NO: 70 - DNA, 67 bp, Homo sapiens
aatacagggattttactcatctccgggctccagtctgaggatgaggctgactattactgtatgattt

SEQ ID NO: 71 - DNA, 67 bp, Homo sapiens
aatgcagggattttactcatctctgggctccagtctgaggatgaggctgactattactgtatgattt

SEQ ID NO: 72 - DNA, 67 bp, Homo sapiens
aatgcagggattttagtcatctctgggctccagtctgaggatgaggctgactattactgtatgattt

SEQ ID NO: 73 - DNA, 67 bp, Homo sapiens
ggtaccaacaaaagccagggaaccctccccggtatctcctgtactaccactcagactccaataaggg

SEQ ID NO: 74 - DNA, 67 bp, Homo sapiens
gtaccagcagaagccagggagctctcccaggttattcctgtatcactactcagactcagacaagcag

SEQ ID NO: 75 - DNA, 67 bp, Homo sapiens
catcgaatggtatcaacagagaccagggaggtccccccagtatataatgaaggttaagagtgatggc

SEQ ID NO: 76 - DNA, 67 bp, Homo sapiens
gaatcggtacctgaccatcaagaacatccaggaagaagatgagagtgactaccactgtggggcagac

SEQ ID NO: 77 - DNA, 67 bp, Homo sapiens
ctacctcaccatctccaacctccagtttgaggatgaggctgattattactgtgagacctgggacagt

SEQ ID NO: 78 - DNA, 71 bp, Homo sapiens
tcatctatggtaacagcaatcggccctcaggggtccctgaccgattctctggctccaagtctggcacctca

SEQ ID NO: 79 - DNA, 70 bp, Homo sapiens
cctgtgctggtcgtctatgatgatagcgaccggccctcagggatccctgagcgattctctggctccaact

SEQ ID NO: 80 - DNA, 72 bp, Homo sapiens
tcaccttgaccatcagcggggcccaggttgaggatgaggctgactattactgttactctgcggctgacaaca

SEQ ID NO: 81 - DNA, 73 bp, Homo sapiens
cagcaaacgaccctccgggatccctgagagattctctggctccagctcagggacaatggccaccttgactatc

SEQ ID NO: 82 - DNA, 71 bp, Homo sapiens
tgtgctggtcatctatagcgatagcaaccggccctcagggatccctgagcgattctctggctccaacccag

SEQ ID NO: 83 - DNA, 73 bp, Homo sapiens
gagaagggccctcggtacttgatgaagcttaacagtgatggcagccacagcaagggggacgggatccctgatc

SEQ ID NO: 84 - DNA, 67 bp, Homo sapiens
ttttaatagtcattccaaatatgagatgcgttgttacaggaagtcccttgccatcctaaaagccacc

SEQ ID NO: 85 - DNA, 67 bp, Homo sapiens
gctgtgactttggcttatctgatcttctgcctgtgttcccttgtgggcattcttcatcttcaaagag

SEQ ID NO: 86 - DNA, 67 bp, Homo sapiens
caatacagaacccattccatttatctttctacagggctgacattgtggcacattcttagagttacca

SEQ ID NO: 87 - DNA, 67 bp, Homo sapiens
gaataacttctatcccagagaggccaaagtacagtggaaggtggataacgccctccaatcgggtaac

SEQ ID NO: 88 - DNA, 70 bp, Mus musculus
ctgggacacagtattctatgaagatcaacagcatgcagcctgaagataccgcaacttatttctgtaaaca

SEQ ID NO: 89 - DNA, 70 bp, Mus musculus
gcaaaaaccttagcagatggtgtgccatcaaggttcagtggcagtggatcaggaacacaatattctctca

SEQ ID NO: 90 - DNA, 70 bp, Mus musculus
agttttctctgaagatcaacagcctgcagcctgaagattttgggagttattactgtcaacatcattatgg

SEQ ID NO: 91 - DNA, 70 bp, Mus musculus
gcaacaaacttagcagatggtgtgccatcaaggttcagtggcagtggatcaggcacacagtattccctca

SEQ ID NO: 92 - DNA, 70 bp, Mus musculus
tttcactctcaccatcagcaatatgcagtctgaagacctggcagattatttctgccagcaatatagcagc

SEQ ID NO: 93 - DNA, 70 bp, Mus musculus
ctctcaccattagcaatgtgcaatctgaagacctggcagattatttctgtctgcaacattggaattatcc

SEQ ID NO: 94 - DNA, 70 bp, Mus musculus
ggtactaatgtagcctggtatcaacagaaaccagggcaatctcctaaagcactgatttactcggcatcct

SEQ ID NO: 95 - DNA, 70 bp, Mus musculus
ggacggatttcactttcaccatcagcagtgtgcaggctgaagacctggcagtttattactgtcagcaaca

SEQ ID NO: 96 - DNA, 70 bp, Mus musculus
cactggggtccccgatcgcttcacaggcagtggatctgcaacagatttcactctgaccatcagcagtgtg

SEQ ID NO: 97 - DNA, 70 bp, Mus musculus
ctctcaccattagcaatgtgcagtctgaagacttggcagattatttctgtcagcaatatagcagctatcc

SEQ ID NO: 98 - DNA, 70 bp, Mus musculus
tatactctcaccatcagcagtgtgcaggctgaagacctggcactttattactgtcagcaacattatagca

SEQ ID NO: 99 - DNA, 70 bp, Mus musculus
accctggggtccctgatcgcttcacaggcagtggatctgcaacagatttcactctgaccatcagcagtct

SEQ ID NO: 100 - DNA, 70 bp, Mus musculus
attcctgcttgtatcagcaggagacagggttaccataacctgcaaggccagtcagagtgtgagtaatgat

SEQ ID NO: 101 - DNA, 70 bp, Mus musculus
caccatctcctgcagagccagtgaaagtgttgaatattatggcacaagtttaatgcagtggtaccaacag

SEQ ID NO: 102 - DNA, 70 bp, Mus musculus
tcaccctcaccattgatcctgtggaggctgatgatgctgcaacctattactgtcagcaaaataatgagga

SEQ ID NO: 103 - DNA, 70 bp, Mus musculus
ctggctatagttatatgcactggtaccaacagaaaccaggacagccacccaaactcctcatctatcttgc

SEQ ID NO: 104 - DNA, 70 bp, Mus musculus
gccaccatctcctgcagagccagcgaaagtgttgataattatggcattagttttatgaactggttccaac

SEQ ID NO: 105 - DNA, 70 bp, Mus musculus
gagccactatcttctgcagagccagccagagtgtcgattataatggaattagttatatgcactggttcca

SEQ ID NO: 106 - DNA, 70 bp, Mus musculus
gccaccatctcctgcaaggccagccaaagtgttgattatgatggtgatagttatatgaactggtaccaac

SEQ ID NO: 107 - DNA, 70 bp, Mus musculus
ctcaccattaatcctgtggaggctgatgatgttgcaacctattactgtcagcaaagtaatgaggatcctc

SEQ ID NO: 108 - DNA, 70 bp, Mus musculus
ctagctatagttatatgcactggtaccaacagaaaccaggacagccacccaaactcctcatcaagtatgc

SEQ ID NO: 109 - DNA, 70 bp, Mus musculus
gacagagggccaccatatcctgccaagccagcgaaagtgtcagttttgctggtacaagtttaatgcactg

SEQ ID NO: 110 - DNA, 70 bp, Mus musculus
agtaagaaggtcaccattagttgcacggccagtgagagcctttattcaagcaaacacaaggtgcactact

SEQ ID NO: 111 - DNA, 70 bp, Mus musculus
gattacactctcagtatcaacagtgtgaagcccgaagatgaaggaatatattactgtcttcaaggttaca

SEQ ID NO: 112 - DNA, 70 bp, Mus musculus
ctctcagtatcaacagtgtggaacctgaagatgttggagtgtattactgtcaaaatggtcacagctttcc

SEQ ID NO: 113 - DNA, 70 bp, Mus musculus
caggagatagcgtcagtctttcctgcagggccagccaaagtattagcaacaacctacactggtatcaaca

SEQ ID NO: 114 - DNA, 70 bp, Mus musculus
ggagatagagtcagtctttcctgcagggccagtcaaagtattagcaactacctacactggtatcaacaaa

SEQ ID NO: 115 - DNA, 70 bp, Mus musculus
ttctcctgcagggccagtcagagcattggcacaagcatacactggtatcagcaaagaacaaatggttctc

SEQ ID NO: 116 - DNA, 70 bp, Mus musculus
gtactggtaccagcagaagtcagatgcctcccccaaactatggatttattacacatccaacctggctcct

SEQ ID NO: 117 - DNA, 70 bp, Mus musculus
ggggctgggatctcttactctctcacaatcagcagcatggaggctgaaaatgatgcaacttattactgcc

SEQ ID NO: 118 - DNA, 70 bp, Mus musculus
tgtctgcatctccaggggaaaaggtcaccatgacctgcagggccagctcaagtgtaagttccagttactt

SEQ ID NO: 119 - DNA, 70 bp, Mus musculus
atcagaatcttttatggagtggaaaccaaaggtactgtttggtctggcaccagtggaaaccagggcaaac

SEQ ID NO: 120 - DNA, 70 bp, Mus musculus
ctctcaccatcagcagtgtgcaggctgaagacctggcagtttattactgtcagaatgattatagttatcc

SEQ ID NO: 121 - DNA, 70 bp, Mus musculus
ggtcactatgagctgcaaatccagtcagagtctgctcaacagtagaacccgaaagaactacttggcttgg

SEQ ID NO: 122 - DNA, 70 bp, Mus musculus
tcctccctggctatgtcagtaggacagaaggtcactatgagctgcaagtccagtcagagccttttaaata

SEQ ID NO: 123 - DNA, 70 bp, Mus musculus
gaaaaggtcactatgagctgtaagtccagtcaaagtgttttatacagttcaaatcagaagaactacttgg

SEQ ID NO: 124 - DNA, 70 bp, Mus musculus
ccgatttcactcttaccatcagcagtgtgcaggctgaagacctggcagtttattactgtcagaatgatca

SEQ ID NO: 125 - DNA, 70 bp, Mus musculus
ccctagctgtgtcagttggagagaaggttactatgagctgcaagtccagtcagagccttttatatagtag

SEQ ID NO: 126 - DNA, 70 bp, Mus musculus
tagctagtggcaaccaaaataactacttggcctggcaccagcagaaaccaggacgatctcctaaaatgct

SEQ ID NO: 127 - DNA, 70 bp, Mus musculus
gagaaacctgggaaaactaataagcttcttatctactctggatccactttgcaatctggaattccatcaa

SEQ ID NO: 128 - DNA, 70 bp, Mus musculus
aactatgccaactgggtccaagaaaaaccagatcatttattcactggtctaataggtggtaccaacaacc

SEQ ID NO: 129 - DNA, 70 bp, Mus musculus
taactatgccaactgggttcaagaaaaaccagatcatttattcactggtctaataggtggtaccagcaac

SEQ ID NO: 130 - DNA, 70 bp, Mus musculus
acagccactcaagcctcctaagtatgtgatggagcttaagaaagatggaagccacagcacaggtgatggg

SEQ ID NO: 131 - DNA, 70 bp, Mus musculus
ctcaagtgtaagttacatgtactggtaccagcagaagccaggatcctcccccaaaccctggatttatcgc

SEQ ID NO: 132 - DNA, 70 bp, Mus musculus
tcacgatcagcagcatggaggctgaagatgttgccacttattactgttttcaggggagtgggtacccact

SEQ ID NO: 133 - DNA, 70 bp, Mus musculus
tattctctcacaatcagcagcatggaggctgaagatgctgccactttttactgccagcagtacagtggtt

SEQ ID NO: 134 - DNA, 70 bp, Mus musculus
actctctcacaatcagcagcatggaggctgaagatgctgcctcttatttctgccatcagtggagtagtta

SEQ ID NO: 135 - DNA, 70 bp, Mus musculus
caatcatgtctgcatctctaggggaggagatcaccctaacctgcagtgccagctcgagtgtaagttacat

SEQ ID NO: 136 - DNA, 70 bp, Mus musculus
cacttctaccaaattctggatttataggacatccaacctggcttcagaagtcccagctcccttcagtggc

SEQ ID NO: 137 - DNA, 70 bp, Mus musculus
tacttgtactggtaccagcagaagtcaggatcctccccaaaactctggatttatagcatatccaacctgg

SEQ ID NO: 138 - DNA, 70 bp, Mus musculus
ctctcacaatcagcagcatggaggctgaagatgctgccacttattactgccaccagtatcatcgttcccc

SEQ ID NO: 139 - DNA, 70 bp, Mus musculus
tacatgtaatggttccagcagaagccaggatcctcccccaaactctggatttatagcatatccaacctgg

SEQ ID NO: 140 - DNA, 70 bp, Mus musculus
catgcactggtaccagcagaagccaggatcctcccccagactctggatttatttaacattcaacttggct

SEQ ID NO: 141 - DNA, 70 bp, Mus musculus
ttgttctctcccagtctccagcaatcctgtctgcatctccaggggagaaggtcacaatgacttgcagggc

SEQ ID NO: 142 - DNA, 70 bp, Mus musculus
gacatctttctctttcacaatcaacagcatggaggctgaagatgttgccacttattactgtcagcaaagg

SEQ ID NO: 143 - DNA, 70 bp, Mus musculus
gccagctcaagtgtaagttacatgcactggttccagcagaagccaggcacttctcccaaactctggattt

SEQ ID NO: 144 - DNA, 70 bp, Mus musculus
gtaagttacatgtactggtaccagcagaagccaagatcctcccccaaaccctggatttatctcacatcca

SEQ ID NO: 145 - DNA, 70 bp, Mus musculus
tgtaagttacatgtacaggtaccagcagaagccaggatcctcacccaaaccctggatttatggcacatcc

SEQ ID NO: 146 - DNA, 70 bp, Mus musculus
gtaagttacatgtactggtaccagcagaagccaggatcctcccccagactcctgatttatgacacatcca

SEQ ID NO: 147 - DNA, 70 bp, Mus musculus
ggtctgagagctcttacactctgacaatcagctgcatgcaggacgaagttgctgccacttactattgtca

SEQ ID NO: 148 - DNA, 70 bp, Mus musculus
ccatgtatgcatctctaggagagagagtcactatcacttgcaaggcgagtcaggacattaatagctattt

SEQ ID NO: 149 - DNA, 70 bp, Mus musculus
cacctatttacattggtacctgcagaagccaggccagtctccaaagctcctgatctacaaagtttccaac

SEQ ID NO: 150 - DNA, 70 bp, Mus musculus
aatggaaacacctatttatattggtacctgcagaagccaggccagtctccaaagctcctgatctacaggg

SEQ ID NO: 151 - DNA, 70 bp, Mus musculus
gtcagagcctcttagatagtgatggaaagacatatttgaattggttgttacagaggccaggccagtctcc

SEQ ID NO: 152 - DNA, 70 bp, Mus musculus
ttacaacagaggcctggccaggctccaaagcacctaatgtatcaggtgtccaaactggaccctggcatcc

SEQ ID NO: 153 - DNA, 70 bp, Mus musculus
atatagtaatggaaaaacctatttgaattggttattacagaggccaggccagtctccaaagcgcctaatc

SEQ ID NO: 154 - DNA, 70 bp, Mus musculus
caggtctagtcagagccttgaaaacagtaatggaaacacctatttgaactggtacctccagaaaccaggc

SEQ ID NO: 155 - DNA, 70 bp, Mus musculus
catccctgtccatggctataggagaaaaagtcaccatcagatgcataaccagcactgatattgatgatga

SEQ ID NO: 156 - DNA, 70 bp, Mus musculus
acattggtagtagcttaaactggcttcagcaggaaccagatggaactattaaacgcctgatctacgccac

SEQ ID NO: 157 - DNA, 70 bp, Mus musculus
cctgtccgtggctacaggagaaaaagtcactatcagatgcataaccagcactgatattgatgatgatatg

SEQ ID NO: 158 - DNA, 70 bp, Mus musculus
gggagacagaataaccatcacttgccaggcaactcaagacattgttaagaatttaaactggtatcagcag

SEQ ID NO: 159 - DNA, 70 bp, Mus musculus
ctctcaccattagcaacctggagcaagaagatattgccacttacttttgccaacagggtaatacgcttcc

SEQ ID NO: 160 - DNA, 70 bp, Mus musculus
ttgcagcagaaaccagggaaatcatttaagggcctgatctatcatggaaccaacttggaagatggagttc

SEQ ID NO: 161 - DNA, 70 bp, Mus musculus
tctgggagaaagtgtcaccatcacatgcctggcaagtcagaccattggtacatggttagcatggtatcag

SEQ ID NO: 162 - DNA, 70 bp, Mus musculus
ccgggcaagtcaggacattcatggttatttaaacttgtttcagcagaaaccaggtgaaactattaaacac

SEQ ID NO: 163 - DNA, 70 bp, Mus musculus
ttctctcaccatcagcaacctggaacctgaagatattgccacttactattgtcagcagtatagtaagctt

SEQ ID NO: 164 - DNA, 70 bp, Mus musculus
ctagtcagagcattgtacatagtaatggaaacacctatttagaatggtacctgcagaaaccaggccagtc

SEQ ID NO: 165 - DNA, 70 bp, Mus musculus
cactcaagatcagcacaataaagcctgaggacttgggaatgtattactgcttacaaggtacacatcagcc

SEQ ID NO: 166 - DNA, 70 bp, Mus musculus
agatcaagcctctatctcttgcaagtctactaagagtcttctgaatagtgatggattcacttatttggac

SEQ ID NO: 167 - DNA, 70 bp, Mus musculus
aaattagtggttacttaagctggcttcagcagaaaccagatggaactattaaacgcctgatctacgccgc

SEQ ID NO: 168 - DNA, 70 bp, Mus musculus
cagtctctcttgtcgggctagtcagggcattagaggtaatttagactggtatcagcagaaaccaggtgga

SEQ ID NO: 169 - DNA, 70 bp, Mus musculus
gcagaaagcagagcaagttccccggctccttatccatagtgcctccactagggccggtggtgtcccagtc

SEQ ID NO: 170 - DNA, 70 bp, Mus musculus
agtattctctcaagatcagtagcctgcatcctgacgatgttgcaacgtattactgtcaaaatgtgttaag

SEQ ID NO: 171 - DNA, 70 bp, Mus musculus
accaacacaagcctggaaaaggtcctaggctgctcatacattacacatctacattacagccaggcatccc

SEQ ID NO: 172 - DNA, 70 bp, Mus musculus
ctccttttctgtatctctaggagacagagtcaccattacttgcaaggcaagtgaggacatatataatcgg

SEQ ID NO: 173 - DNA, 70 bp, Mus musculus
ctacttgtctgtatctctaggaggcagagtcaccattacttgcaaggcaagtgaccacattaataattgg

SEQ ID NO: 174 - DNA, 70 bp, Mus musculus
ccaggaaatattcctaaactattgatctataaggcttccaacttgcacacaggcgtcccatcaaggttta

SEQ ID NO: 175 - DNA, 70 bp, Mus musculus
tattgtgatgacgcaggctgcattctccaatccagtcactcttggaacatcagcttccatctcctgcagg

SEQ ID NO: 176 - DNA, 70 bp, Mus musculus
atattgtgatgactcaggctgcaccctctgtacctgtcactcctggagagtcagtatccatctcctgcag

SEQ ID NO: 177 - DNA, 70 bp, Mus musculus
tctagtaagagtctcctatataaggatgggaagacatacttgaattggtttctgcagagaccaggacaat

SEQ ID NO: 178 - DNA, 70 bp, Mus musculus
tgacttgccaggcaagtcagggcactagcattaatttaaactggtttcagcaaaaaccagggaaagctcc

SEQ ID NO: 179 - DNA, 70 bp, Mus musculus
aattgtgctcactcagtctccagccatcacagctgcatctctggggcaaaaggtcaccatcacctgcagt

SEQ ID NO: 180 - DNA, 70 bp, Mus musculus
cccggggagaagatcactatcacctgcagtgccagctcaagtataagttccaattacttgcattggtatc

SEQ ID NO: 181 - DNA, 70 bp, Mus musculus
gctcaagtataagttccagcaacttgcactggtaccagcagaagtcagaaacctcccccaaaccctggat

SEQ ID NO: 182 - DNA, 70 bp, Mus musculus
ttacttgcactggtaccagcagaagtcaggcgcttcccccaaacccttgattcataggacatccaacctg

SEQ ID NO: 183 - DNA, 70 bp, Mus musculus
ctcaagtgtaagttacatgcactggtaccagcagaagtcaggcacctcccccaaaagatggatttatgac

SEQ ID NO: 184 - DNA, 70 bp, Mus musculus
aggatcctcgcccaaaccctggatttatgacacatccaacctggcttctggattccctgctcgcttcagt

SEQ ID NO: 185 - DNA, 70 bp, Mus musculus
ccagctcaagtataagttacatgcactggtaccagcagaagccaggcacctcccccaaaagatggattta

SEQ ID NO: 186 - DNA, 70 bp, Mus musculus
cttcacccattgtcaagagcttcaacaggaatgagtgttagagacaaaggtcctgagacgccaccaccag

SEQ ID NO: 187 - DNA, 70 bp, Mus musculus
agaaacatgcccaagtgtatccttggtgcttttgcctaccatagcccttctctctaccctcaaaatgcac

SEQ ID NO: 188 - DNA, 70 bp, Mus musculus
ccgtgtttccaccttcctctgaggagctcaaggaaaacaaagccacactggtgtgtctgatttccaactt

SEQ ID NO: 189 - DNA, 70 bp, Mus musculus
aatcccttctttcattcacacaggtcagcccaagtccactcccacactcaccatgtttccaccttcccct

SEQ ID NO: 190 - DNA, 70 bp, Mus musculus
cgtgcaccgcaagtgcttctaggcggactgttactgagctgcgttttacaccctttctttgacaaaacct

SEQ ID NO: 191 - DNA, 70 bp, Homo sapiens
aggccaatctccaaggcgcctaatttataaggtttctaactgggactctggggtcccagacagattcagc

SEQ ID NO: 192 - DNA, 70 bp, Homo sapiens
agctacttagcctggtaccagcagaaacctggcctggcgcccaggctcctcatctatgatgcatccagca

SEQ ID NO: 193 - DNA, 70 bp, Homo sapiens
tcctctctgtgactccaggggagaaagtcaccatcacctgccaggccagtgaaggcattggcaactactt

SEQ ID NO: 194 - DNA, 70 bp, Homo sapiens
aggccaatctccaaggcgcctaatttataaggtttctaaccgggactctggggtcccagacagattcagc

SEQ ID NO: 195 - DNA, 70 bp, Homo sapiens
ctatttgtattggtacctgcagaagccaggccagtctccacagctcctaatctatgaagtttccagccgg

SEQ ID NO: 196 - DNA, 70 bp, Homo sapiens
tatttgggttctaatcgggcctccggggtccctgacaggttcagtggcagtggatcaggcacagatttta

SEQ ID NO: 197 - DNA, 70 bp, Homo sapiens
atttgtattggtacctgcagaagccaggccagcctccacagctcctgatctatgaagtttccaaccggtt

SEQ ID NO: 198 - DNA, 70 bp, Homo sapiens
aaagttcctaagctcctgatctatgctgcatccactttgcaatcaggggtcccatctcggttcagtggca

SEQ ID NO: 199 - DNA, 70 bp, Homo sapiens
tccaagactcctaatttataagatttctaaccggttctctggggtcccagacagattcagtggcagtggg

SEQ ID NO: 200 - DNA, 70 bp, Homo sapiens
ctgccgggccagtcagagcattggtagtagcttacactggtaccagcagaaaccagatcagtctccaaag

SEQ ID NO: 201 - DNA, 70 bp, Homo sapiens
aaattgtgttgacgcagtctccaggcaccctgtctttgtctccaggggaaagagccaccctctcctgcag

SEQ ID NO: 202 - DNA, 70 bp, Homo sapiens
ttagaaatgatttaggctggtatcagcagaaaccagggaaagcccctaagcgcctgatctatgctgcatc

SEQ ID NO: 203 - DNA, 70 bp, Homo sapiens
atacacctatttgtattggtttctgcagaaagccaggccagtctccacactcctgatctatgaagtttcc

SEQ ID NO: 204 - DNA, 70 bp, Homo sapiens
gatttcacactgaaaatcagcagggtggaagctgaggatgtcggggtttattactgcacgcaagctacac

SEQ ID NO: 205 - DNA, 70 bp, Homo sapiens
ccctcacaattaataacatagaatctgaggatgctgcatattacttctgtctacaacatgataatttccc

SEQ ID NO: 206 - DNA, 70 bp, Homo sapiens
tccctggctgtgtctctgggcgagagggccaccatcaactgcaagtccagccagagtgttttatacagct

SEQ ID NO: 207 - DNA, 70 bp, Homo sapiens
ggcgagtcagggcattagcaattatttagcctggtttcagcagaaaccagggaaagcccctaagtccctg

SEQ ID NO: 208 - DNA, 70 bp, Homo sapiens
cagctacttaacctggtatcagcagaaacctggccaggcgcccaggctcctcatctatggtgcatccacc

SEQ ID NO: 209 - DNA, 70 bp, Homo sapiens
tcaccatcagcagcctgcagcctgaagattttgcaacttattactgtctacaagattacaattaccctcc

SEQ ID NO: 210 - DNA, 70 bp, Homo sapiens
agacagagtcaccatcacttgccgggccagtcagagtattagtagctggttggcctggtatcagcagaaa

SEQ ID NO: 211 - DNA, 70 bp, Homo sapiens
tcgggcgaggcagggcattagcaattatttagcctggtttcagcagaaaccagggaaagtccctaagcac

SEQ ID NO: 212 - DNA, 70 bp, Homo sapiens
ctggttagcctggtatcagcagaaaccagagaaagcccctaagtccctgatctatgctgcatccagtttg

SEQ ID NO: 213 - DNA, 70 bp, Homo sapiens
tcaccatcagcagcctgcagtctgaagattttgcagtttattactgtcagcagtataataactgacctcc

SEQ ID NO: 214 - DNA, 70 bp, Homo sapiens
ctcaccatcagcagcctgcagcctgaagattttgcaacttattactgtcaacagtttaataattaccctc

SEQ ID NO: 215 - DNA, 70 bp, Homo sapiens
agatttcactctcactatcagcagcctgcagcctgaagattttgcaacttactattgtcaacaggctaac

SEQ ID NO: 216 - DNA, 70 bp, Homo sapiens
tcaccatcagcagcctgcagtctgaagattttgcagtttattactgtcagcagtataataactggcctcc

SEQ ID NO: 217 - DNA, 70 bp, Homo sapiens
gtgggcctgggacagacttcactctcaccatcagcagcctagagcctgaagattttgcagtttattactg

SEQ ID NO: 218 - DNA, 70 bp, Homo sapiens
gaaaccagggaaatcccctaagctcttcctctatgatgcaaaagatttgcaccctggggtctcatcgagg

SEQ ID NO: 219 - DNA, 70 bp, Homo sapiens
gtatcagcaaaaaccagcaaaagcccctaagctcttcatctattatgcatccagtttgcaaagtggggtc

SEQ ID NO: 220 - DNA, 70 bp, Homo sapiens
tccttactctctgcatctacaggagacagagtcaccatcagttgtcggatgagtcagggcattagcagtt

SEQ ID NO: 221 - DNA, 70 bp, Homo sapiens
cagctacttatcctggtaccagcagaaacctgggcaggctcccaggctcctcatctatggtgcatccacc

SEQ ID NO: 222 - DNA, 70 bp, Homo sapiens
ctcaccatcagcagcctgcagcctgaagattttgcaacttattactgtcaacagtttaatagttaccctc

SEQ ID NO: 223 - DNA, 70 bp, Homo sapiens
gatctgggacagatttcactctcaccatcagcagcctgcagcctgaagattttgcaacttactattgtca

SEQ ID NO: 224 - DNA, 70 bp, Homo sapiens
aaagagccaccctctcctgcagggccagtcagagtgttagcagctacttagcctggtaccaacagaaacc

SEQ ID NO: 225 - DNA, 70 bp, Homo sapiens
tctcacaatcagcagcctgcagcctgaagattttgcaacttattactgtcaacagcttaatagttaccct

SEQ ID NO: 226 - DNA, 70 bp, Homo sapiens
cattctctgcatctacaggagacagagtcaccatcacttgtcgggcgagtcagggtattagcagttattt

SEQ ID NO: 227 - DNA, 70 bp, Homo sapiens
atctatacgctttcctatcgggcctctggagtcccagacaggttcagtggcagtgggtcaggcactgatt

SEQ ID NO: 228 - DNA, 70 bp, Homo sapiens
tctcaccatcagcagtctgcaacctgaagattttgcaacttactactgtcaacagagttacagtacccct

SEQ ID NO: 229 - DNA, 70 bp, Homo sapiens
tcactatcagcagcctgcagcctgaagatgttgcaacttattacggtcaacggacttacaatgcccctcc

SEQ ID NO: 230 - DNA, 70 bp, Homo sapiens
acgatgcatccaatttggaaacaggggtcccatcaaggttcagtggaagtggatctgggacagattttac

SEQ ID NO: 231 - DNA, 70 bp, Homo sapiens
caggaaaggctcccaaactcctcatctattatgatgatctgctgccctcaggggtctctgaccgattctc

SEQ ID NO: 232 - DNA, 70 bp, Homo sapiens
atcactgggctccaggctgaggatgaggctgattattactgccagtcctatgacagcagcctgagtggtt

SEQ ID NO: 233 - DNA, 70 bp, Homo sapiens
gtcaccatctcttgttctggaagcagctccaacatcggaagtaatactgtaaactggtaccagcagctcc

SEQ ID NO: 234 - DNA, 70 bp, Homo sapiens
aattatgtatactggtaccagcagctcccaggaacggcccccaaactcctcatctatagtaataatcagc

SEQ ID NO: 235 - DNA, 70 bp, Homo sapiens
ccatcactggactccagtctgaggatgaggctgattattactgcaaagcatgggataacagcctgaatgc

SEQ ID NO: 236 - DNA, 70 bp, Homo sapiens
atcaccggactccagactggggacgaggccgattattactgcggaacatgggatagcagcctgagtgctg

SEQ ID NO: 237 - DNA, 70 bp, Homo sapiens
cgtctctgggctccaggctgaggatgaggctgattattactgcagctcatatgcaggcagcaacaatttc

SEQ ID NO: 238 - DNA, 70 bp, Homo sapiens
accaaggagcagcttggctgcagcagcaccagggccaccctcccaaactcctatcctacaggaataacaa

SEQ ID NO: 239 - DNA, 70 bp, Homo sapiens
tctccggggaagacggtaaccatctcctgcacccgcagcagtggcagcattgccagcaactatgtgcagt

SEQ ID NO: 240 - DNA, 70 bp, Homo sapiens
catctctgggctccaggctgaggatgaggctgattattactgctgctcatatgcaggcagctacactttc

SEQ ID NO: 241 - DNA, 70 bp, Homo sapiens
catgatttatgaggtcagtaatcggccctcaggggtttctaatcgcttctctggctccaagtctggcaac

SEQ ID NO: 242 - DNA, 70 bp, Homo sapiens
ttggtagttataaccgtgtctcctggtaccagcagcccccaggcacagcccccaaactcatgatttatga

SEQ ID NO: 243 - DNA, 70 bp, Homo sapiens
aatctctgggctccaggctgaggacgaggctgattattactgctgctcatatgcaggtagtagcactttc

SEQ ID NO: 244 - DNA, 70 bp, Homo sapiens
cgttggggattatgatcatgtcttctggtaccaaaagcgtctcagcactacctccagactcctgatttac

SEQ ID NO: 245 - DNA, 70 bp, Homo sapiens
tccgtgtccccaggacagacagccagcatcacctgctctggagataaattgggggataaatatgcttgct

SEQ ID NO: 246 - DNA, 70 bp, Homo sapiens
aaaaaatatgcttattggtaccagcagaagccaggccagttccctgtgctggtgatatataaagacagcg

SEQ ID NO: 247 - DNA, 70 bp, Homo sapiens
acagacagtcaggatcacatgccaaggagacagcctcagaagctattatgcaagctggtaccagcagaag

SEQ ID NO: 248 - DNA, 70 bp, Homo sapiens
catcagcagggtcgaagccggggatgaggccgactattactgtcaggtgtgggatagtagtagtgatcat

SEQ ID NO: 249 - DNA, 70 bp, Homo sapiens
ccctgagttggtgatatacgaagatagtgagcggtaccctggaatccctgaacgattctctgggtccacc

SEQ ID NO: 250 - DNA, 70 bp, Homo sapiens
acagtcacgttgaccatcagtggagtccaggcagaagatgaggctgactattactgtcaatcagcagaca

SEQ ID NO: 251 - DNA, 70 bp, Homo sapiens
ccttgaccatcagcggggcccaggttgaggatgaggctgactattactgttactctgcggctgacaacaa

SEQ ID NO: 252 - DNA, 70 bp, Homo sapiens
gaccatcagcagagcccaagccggggatgaggctgactattactgtcaggtgtgggacagcagcactgca

SEQ ID NO: 253 - DNA, 70 bp, Homo sapiens
tcagtggggcccaggtggaggatgaagctgactactactgttactcaacagacagcagtggtaatcatag

SEQ ID NO: 254 - DNA, 70 bp, Homo sapiens
tccaacccagggaacaccgccaccctaaccatcagcaggatcgaggctggggatgaggctgactattact

SEQ ID NO: 255 - DNA, 70 bp, Homo sapiens
gacactgtcaggtgtgcagcctgaggacgaggctgagtattactgcctgctctactatggtggtgctcag

SEQ ID NO: 256 - DNA, 70 bp, Homo sapiens
tcattatccctactggttccagcagaagcctggccaagcccccaggacactgatttatgatacaagcaac

SEQ ID NO: 257 - DNA, 70 bp, Homo sapiens
gaacaaagctgccctcaccatcacgggggcccaggcagatgatgaatctgattattactgtgtgctgtat

SEQ ID NO: 258 - DNA, 70 bp, Homo sapiens
accttcctcctccgcatctcctggagaatccgccagactcacctgcaccttgcccagtgacatcaatgtt

SEQ ID NO: 259 - DNA, 70 bp, Homo sapiens
gtaccagcagaagccagggagtcctccccagtatctcctgaggtacaaatcagactcagataagcagcag

SEQ ID NO: 260 - DNA, 70 bp, Homo sapiens
gtaccagcagaagccagagagccctccccggtatctcctgagctactactcagactcaagtaagcatcag

SEQ ID NO: 261 - DNA, 70 bp, Homo sapiens
atgctgagcagtggcttcagtgttggggacttctggataaggtggtaccaacaaaagccagggaaccctc

SEQ ID NO: 262 - DNA, 70 bp, Homo sapiens
acctggggtccccagtcgagtctctggctccaaggagacctcaagtaacacagcgtttttgctcatctct

SEQ ID NO: 263 - DNA, 70 bp, Homo sapiens
tgagcacagcacctacaccatcgaatggtatcaacagagaccagggaggtccccccagtatataatgaag

SEQ ID NO: 264 - DNA, 70 bp, Homo sapiens
atcaagaacatccaggaagaagatgagagtgactaccactgtggggcagaccatggcagtgggagcaact

SEQ ID NO: 265 - DNA, 70 bp, Homo sapiens
ccagggaaggcccctcggtacttgatgaagcttgaaggtagtggaagctacaacaaggggagcggagttc

SEQ ID NO: 266 - DNA, 70 bp, Homo sapiens
gagaagggccctcggtacttgatgaagcttaacagtgatggcagccacagcaagggggacgggatccctg

SEQ ID NO: 267 - DNA, 67 bp, Mus musculus
catgtcgagcaagtgagaacatttactacagtttagcatggtatcagcagaagcaagggaaatctcc

SEQ ID NO: 268 - DNA, 67 bp, Mus musculus
ttcacaattatttagcatggtatcagcagaaacagggaaaatctcctcagctcctggtctataatgc

SEQ ID NO: 269 - DNA, 67 bp, Mus musculus
cgagcaagtgagaatatttacagttatttagcatggtatcagcagaaacagggaaaatctcctcagc

SEQ ID NO: 270 - DNA, 67 bp, Mus musculus
cgagcaagtgagaatatttacagtaatttagcatggtatcagcagaaacagggaaaatctcctcagc

SEQ ID NO: 271 - DNA, 67 bp, Mus musculus
tggtatcaacagaaaccaggacaatctcctaaactactgatttactcggcatccaatcggtacactg

SEQ ID NO: 272 - DNA, 67 bp, Mus musculus
gatctgggacagatttcactctcaccattagcaatgtgcaatctgaagacctggcagattatttctg

SEQ ID NO: 273 - DNA, 67 bp, Mus musculus
ctcaccatcagcaatgtgcagtctgaagacttggcagagtatttctgtcagcaatataacagctatc

SEQ ID NO: 274 - DNA, 67 bp, Mus musculus
ctttcaccatcagcagtgtgcaggctgaagacctggcagtttattactgtcagcaacattatagtac

SEQ ID NO: 275 - DNA, 67 bp, Mus musculus
caaggccagtgagaatgtgggtacttatgtatcctggtatcaacagaaaccagagcagtctcctaaa

SEQ ID NO: 276 - DNA, 70 bp, Mus musculus
cagatttcactctcaccattagcaatgtgcagtctgaagacttggcagattatttctgtcagcaatatag

SEQ ID NO: 277 - DNA, 69 bp, Mus musculus
attatactctcaccatcagcagtgtgcaggctgaagacctggcactttattactgtcagcaacattata

SEQ ID NO: 278 - DNA, 67 bp, Mus musculus; identical to SEQ ID NO: 275

SEQ ID NO: 279 - DNA, 67 bp, Mus musculus
cagtctcctaaactgctgatatactatgcatccaatcgctacactggagtccctgatcgcttcactg

SEQ ID NO: 280 - DNA, 67 bp, Mus musculus
gtaccaacagaaaccaggacagccacccaaactcctcatctatgctgcatccaacgtagaatctggg

SEQ ID NO: 281 - DNA, 67 bp, Mus musculus
gtctaggacagacttcaccctcaccattgatcctgtggaggctgatgatgctgcaacctattactgt

SEQ ID NO: 282 - DNA, 67 bp, Mus musculus
gtaccaacagaaaccaggacagccacccaaactcctcatctatcttgcatccaacctagaatctggg

SEQ ID NO: 283 - DNA, 67 bp, Mus musculus
ctcaacatccatcctatggaggaggatgatactgcaatgtatttctgtcagcaaagtaaggaggttc

SEQ ID NO: 284 - DNA, 67 bp, Mus musculus
ctgcagagccagccagagtgtcgattataatggaattagttatatgcactggttccaacagaaacca

SEQ ID NO: 285 - DNA, 67 bp, Mus musculus
gtaccaacagaaaccaggacagccacccaaactcctcatctatgctgcatccaatctagaatctggg

SEQ ID NO: 286 - DNA, 67 bp, Mus musculus
ttcagtggcagtgggtctaggacagacttcaccctcaccattaatcctgtggaggctgatgatgttg

SEQ ID NO: 287 - DNA, 67 bp, Mus musculus
ctcaacatccatcctgtggaggaggaggatactgcaacatattactgtcagcacagttgggagattc

SEQ ID NO: 288 - DNA, 67 bp, Mus musculus
ctgagtcagacttcactctcaccatcgatcctgtggaggaagatgatgctgcaatgtattactgtat

SEQ ID NO: 289 - DNA, 67 bp, Mus musculus
tctgggacagatttcactctgaccatcagcagtgtacaggttgaagacctcacacattattactgtg

SEQ ID NO: 290 - DNA, 71 bp, Mus musculus
gagtatttacaagaacctacactggtatcaacagaaatcacatcggtctccaaggcttctcatcaagtatg

SEQ ID NO: 291 - DNA, 67 bp, Mus musculus
gggtcagatttcactctcagtatcaacagtgtggaacctgaagatgttggagtgtattactgtcaaa

SEQ ID NO: 292 - DNA, 68 bp, Mus musculus
tggatcagggacagatttcactctcagtatcaacagtgtggagactgaagattttggaatgtatttct

SEQ ID NO: 293 - DNA, 67 bp, Mus musculus
agtcaaagtattagcaactacctacactggtatcaacaaaaatcacatgagtctccaaggcttctca

SEQ ID NO: 294 - DNA, 67 bp, Mus musculus
tttagtggcagtggatcagggacagattttactcttagcatcaacagtgtggagtctgaagatattg

SEQ ID NO: 295 - DNA, 67 bp, Mus musculus
taccagcagaagtcagatgcctcccccaaactatggatttattacacatccaacctggctcctggag

SEQ ID NO: 296 - DNA, 67 bp, Mus musculus
gatctcttactctctcacaatcagcagcatggaggctgaaaatgatgcaacttattactgccagcag

SEQ ID NO: 297 - DNA, 67 bp, Mus musculus
ctcttactctctcacaatcagcagtgtggaggctgaagatgctgccacttattactgccagcagtac

SEQ ID NO: 298 - DNA, 67 bp, Mus musculus
catctgataggtactctggagtccctgatcgtttcataggcagtggatctgtgacagatttcactct

SEQ ID NO: 299 - DNA, 67 bp, Mus musculus
gagagaaggtcactatgagctgcaagtccagtcagagtctgttaaacagtggaaatcaaaagaacta

SEQ ID NO: 300 - DNA, 67 bp, Mus musculus
gaaggtcactatgagctgcaaatccagtcagagtctgctcaacagtagaacccgaaagaactacttg

SEQ ID NO: 301 - DNA, 67 bp, Mus musculus
gacagaaggtcactatgagctgcaagtccagtcagagccttttaaatagtagcaatcaaaagaacta

SEQ ID NO: 302 - DNA, 69 bp, Mus musculus
gcaggagaaaaggtcactatgagctgtaagtccagtcaaagtgttttatacagttcaaatcagaagaac

SEQ ID NO: 303 - DNA, 67 bp, Mus musculus; identical to SEQ ID NO: 299

SEQ ID NO: 304 - DNA, 73 bp, Mus musculus
ttggagagaaggttactatgagctgcaagtccagtcagagccttttatatagtagcaatcaaaagaactactt

SEQ ID NO: 305 - DNA, 67 bp, Mus musculus
gatctcctaaaatgctgataatttgggcatccactagggtatctggagtccctgatcgcttcatagg

SEQ ID NO: 306 - DNA, 73 bp, Mus musculus
tctcctggagaaaccattactattaattgcagggcaagtaagagcattagcaaatatttagcctggtatcaag

SEQ ID NO: 307 - DNA, 67 bp, Mus musculus
caaattgttctcacccagtctccagcaatcatgtctgcatctccaggggagaaggtcaccatatcct

SEQ ID NO: 308 - DNA, 67 bp, Mus musculus
ctggaaactcttactctctcacgatcagcagcatggaggctgaagatgttgccacttattactgttt

SEQ ID NO: 309 - DNA, 67 bp, Mus musculus
caatcatgtctgcatctcctggggagaaggtcaccatgacctgcagtgccagatcaagtgtaagttc

SEQ ID NO: 310 - DNA, 67 bp, Mus musculus
caaattgttctcacccagtctccagcaatcatgtctgcatctcctggggagaaggtcaccttgacct

SEQ ID NO: 311 - DNA, 67 bp, Mus musculus
cacttctcccaaactcttgatttatagcacatccaacctggcttctggagtcccttctcgcttcagt

SEQ ID NO: 312 - DNA, 67 bp, Mus musculus
cttctaccaaattctggatttataggacatccaacctggcttcagaagtcccagctcccttcagtgg

SEQ ID NO: 313 - DNA, 67 bp, Mus musculus
gatcctccccaaaactctggatttatagcatatccaacctggcttctggagtcccagctcgcttcag

SEQ ID NO: 314 - DNA, 67 bp, Mus musculus
caaattgttctcacccagtctccagcaatcatgtctgcatctctaggggaacgggtcaccatgacct

SEQ ID NO: 315 - DNA, 67 bp, Mus musculus
gatcctcccccaaactctggatttatagcatatccaacctggcttctggagtccctgctcgcttcag

SEQ ID NO: 316 - DNA, 67 bp, Mus musculus
ctctggatttatttaacattcaacttggcttctggagtccctgctcgcttcagtggcagtgggtctg

SEQ ID NO: 317 - DNA, 67 bp, Mus musculus
ctctcacaatcagcagagtggaggctgaagatgctgccacttattactgccagcagtggagtagtaa

SEQ ID NO: 318 - DNA, 67 bp, Mus musculus
catctttctctttcacaatcaacagcatggaggctgaagatgttgccacttattactgtcagcaaag

SEQ ID NO: 319 - DNA, 67 bp, Mus musculus
caagtgtaagttacatgcactggttccagcagaagccaggcacttctcccaaactctggatttatag

SEQ ID NO: 320 - DNA, 67 bp, Mus musculus
catctccaggggagaaggtcaccatgacctgcagtgccagctcaagtgtaagttacatgtactggta

SEQ ID NO: 321 - DNA, 67 bp, Mus musculus
cttcagtggcagtggatctgggacctcttattctctcacaatcagcagcatggaggctgaagatgct

SEQ ID NO: 322 - DNA, 67 bp, Mus musculus; identical to SEQ ID NO: 320

SEQ ID NO: 323 - DNA, 67 bp, Mus musculus
gaggggagaaggtcaccatcacctgccgtgccagctcaagtataagttccaattacttacactggta

SEQ ID NO: 324 - DNA, 68 bp, Mus musculus
tggatctgggcaagattattctctcaccatcagcagcctggagtatgaagatatgggaatttattatt

SEQ ID NO: 325 - DNA, 67 bp, Mus musculus
catctcttgcagatctagtcagagccttgtacacagtaatggaaacacctatttacattggtacctg

SEQ ID NO: 326 - DNA, 68 bp, Mus musculus
ccatctcttgcagatctagtcagagccttgtacacagtaatggaaacacctatttatattggtacctg

SEQ ID NO: 327 - DNA, 67 bp, Mus musculus
ctcttagatagtgatggaaagacatatttgaattggttgttacagaggccaggccagtctccaaagc

SEQ ID NO: 328 - DNA, 68 bp, Mus musculus
aaactggaccctggcatccctgacaggttcagtggcagtggatcagaaacagattttacacttaaaat

SEQ ID NO: 329 - DNA, 67 bp, Mus musculus
ctatttgaattggttattacagaggccaggccagtctccaaagcgcctaatctatctggtgtctaaa

SEQ ID NO: 330 - DNA, 67 bp, Mus musculus
ctccatctcttgcaggtctagtcagagccttgaaaacagtaatggaaacacctatttgaactggtac

SEQ ID NO: 331 - DNA, 67 bp, Mus musculus
ggaacctcctaagctccttatttcagaaggcaatactcttcgtcctggagtcccatcccgattctcc

SEQ ID NO: 332 - DNA, 67 bp, Mus musculus
ggacattggtagtagcttaaactggcttcagcaggaaccagatggaactattaaacgcctgatctac

SEQ ID NO: 333 - DNA, 67 bp, Mus musculus; identical to SEQ ID NO: 331

SEQ ID NO: 334 - DNA, 67 bp, Mus musculus
tctgggagacagaataaccatcacttgccaggcaactcaagacattgttaagaatttaaactggtat

SEQ ID NO: 335 - DNA, 67 bp, Mus musculus
ttcagtggcagtgggtctggaacagattattctctcaccattagcaacctggagcaagaagatattg

SEQ ID NO: 336 - DNA, 67 bp, Mus musculus
cagcagaaaccagggaaatcatttaagggcctgatctatcatggaaccaacttggaagatggagttc

SEQ ID NO: 337 - DNA, 67 bp, Mus musculus
gaccattggtacatggttagcatggtatcagcagaaaccagggaaatctcctcagctcctgatttat

SEQ ID NO: 338 - DNA, 67 bp, Mus musculus
acctgatctatgaaacatccaatttagattctggtgtcccaaaaaggttcagtggcagtaggtctgg

SEQ ID NO: 339 - DNA, 67 bp, Mus musculus
ttattctctcaccatcagcaacctggaacctgaagatattgccacttactattgtcagcagtatagt

SEQ ID NO: 340 - DNA, 73 bp, Mus musculus
ggagatcaagcctccatctcttgcagatctagtcagagcattgtacatagtaatggaaacacctatttagaat

SEQ ID NO: 341 - DNA, 67 bp, Mus musculus
gatttcacactcaagatcagcacaataaagcctgaggacttgggaatgtattactgcttacaaggta

SEQ ID NO: 342 - DNA, 67 bp, Mus musculus
ctcaagatcagcagagtggaggctgaggatttgggagtttattattgcttccagagtaactatcttc

SEQ ID NO: 343 - DNA, 67 bp, Mus musculus
ggaaattagtggttacttaagctggcttcagcagaaaccagatggaactattaaacgcctgatctac

SEQ ID NO: 344 - DNA, 68 bp, Mus musculus
ctagtcagggcattagaggtaatttagactggtatcagcagaaaccaggtggaactattaaactcctg

SEQ ID NO: 345 - DNA, 68 bp, Mus musculus
gaaacaacacaggctccagcttctctgagtttttctcttggtgaaacagcaacactgtcatgcaggtc

SEQ ID NO: 346 - DNA, 67 bp, Mus musculus
caagtgagaatatttacggtgctttaaattggtatcagcggaaacagggaaaatctcctcagctcct

SEQ ID NO: 347 - DNA, 67 bp, Mus musculus
ctgctcatacattacacatctacattacagccaggcatcccatcaaggttcagtggaagtgggtctg

SEQ ID NO: 348 - DNA, 67 bp, Mus musculus
gttccttcaagattcagtggcagtggatctggaaaggattacactctcagcattaccagtcttcaga

SEQ ID NO: 349 - DNA, 67 bp, Mus musculus
gacatccagatgacacaatcttcatcctacttgtctgtatctctaggaggcagagtcaccattactt

SEQ ID NO: 350 - DNA, 67 bp, Mus musculus
ggaaatattcctaaactattgatctataaggcttccaacttgcacacaggcgtcccatcaaggttta

SEQ ID NO: 351 - DNA, 67 bp, Mus musculus
gatattgtgatgacgcaggctgcattctccaatccagtcactcttggaacatcagcttccatctcct

SEQ ID NO: 352 - DNA, 68 bp, Mus musculus
ctgagaatcagtagagtggaggctgaggatgtgggtgtttattactgtatgcaacatctagaatatcc

SEQ ID NO: 353 - DNA, 67 bp, Mus musculus
ctggaaatcagtagagtgaaggctgaggatgtgggtgtgtattactgtcaacaacttgtagagtatc

SEQ ID NO: 354 - DNA, 67 bp, Mus musculus
tctcaccatcagcagcctggaggatgaagatatggcaacttatttctgtctacagcatagttatctc

SEQ ID NO: 355 - DNA, 67 bp, Mus musculus
catggatttatgaaatatccaaactggcttctggagtcccagctcgcttcagtggcagtgggtctgg

SEQ ID NO: 356 - DNA, 67 bp, Mus musculus
caattacttgcattggtatcagcagaagccaggattctcccctaaactcttgatttataggacatcc

SEQ ID NO: 357 - DNA, 67 bp, Mus musculus
cttctggagtccctgttcgcttcagtggcagtggatctgggacctcttattctctcacaatcagcag

SEQ ID NO: 358 - DNA, 67 bp, Mus musculus
ggtctgggacctcttactctctcacaatcagcagcgtggaggctgaagatgatgcaacttattactg

SEQ ID NO: 359 - DNA, 67 bp, Mus musculus
cccaaaagatggatttatgacacatccaaactggcttctggagtccctgctcgcttcagtggcagtg

SEQ ID NO: 360 - DNA, 67 bp, Mus musculus
caaattcttctcacccagtctccagcaatcatgtctgcatctccaggggagaaggtcaccatgacct

SEQ ID NO: 361 - DNA, 67 bp, Mus musculus; identical to SEQ ID NO: 359

SEQ ID NO: 362 - DNA, 67 bp, Mus musculus; identical to SEQ ID NO: 321

SEQ ID NO: 363 - DNA, 67 bp, Mus musculus
aagtactggggctgttacaactagtaactatgccaactgggtccaagaaaaaccagatcatttattc

SEQ ID NO: 364 - DNA, 68 bp, Mus musculus
gtactggggctgttacaactagtaactatgccaactgggttcaagaaaaaccagatcatttattcact

SEQ ID NO: 365 - DNA, 67 bp, Mus musculus
ttagcatttccaacatccagcctgaagatgaagcaatatacatctgtggtgtgggtgatacaattaa

SEQ ID NO: 366 - DNA, 24 bp, Homo sapiens
tctgttcaagccgaggcatcctta

SEQ ID NO: 367 - DNA, 24 bp, Homo sapiens
tccaaagcgtgttcccttcggtat

(19) United States
(12) Patent Application Publication
(10) Pub. No.: US 2008/0003605 A1
(43) Pub. Date: Jan. 3, 2008

(54) MICROARRAY ANALYSIS OF LIGHT CHAIN VARIABLE GENE EXPRESSION AND METHODS OF USE

(75) Inventors: Martin Weigert, Chicago, IL (US); Nathan Schoettler, Chicago, IL (US); Dongyao Ni, Chicago, IL (US)

Correspondence Address: MICHAEL BEST & FRIEDRICH LLP, ONE SOUTH PINCKNEY STREET, P O BOX 1806, MADISON, WI, 53701, US

(73) Assignee: THE UNIVERSITY OF CHICAGO, Chicago, IL (US)

(21) Appl. No.: 11/753,263

(22) Filed: May 24, 2007

Related U.S. Application Data
(60) Provisional application No. 60/803,099, filed on May 24, 2006.

(51) Int. Cl. C12Q 1/68

(52) U.S. Cl. 435/6

(57) ABSTRACT

Disclosed are microarrays comprising a plurality of oligonucleotide species capable of hybridizing to a polynucleotide comprising a sequence encoding at least a portion of a light chain variable region or a complement thereof. Also disclosed are methods of identifying light chain variable genes associated with a disease, methods of diagnosing a disease and methods of monitoring a disease. Methods of evaluating the ability of a therapeutic agent or a treatment to alter expression of the light chain variable gene are also provided.

### CROSS-REFERENCE TO RELATED APPLICATIONS

This application claims priority to U.S. Provisional Application No. 60/803,099 filed on May 24, 2006, which is incorporated by reference in its entirety.

### STATEMENT REGARDING FEDERALLY SPONSORED RESEARCH

Not applicable.

### INTRODUCTION

Immunoglobulins are comprised of a heavy chain and a light chain. Both heavy chains and light chains are encoded by a series of gene segments that are rearranged by genomic recombination events that occur during B cell development. The resulting immunoglobulins are expressed on the cell surface as B cell receptors and may be secreted as antibodies. The genomic recombination events cause expression patterns of the various immunoglobulin gene segments to vary from one individual to another.

There are numerous pathologic conditions caused by the formation of auto-antibodies, which recognize self-antigens. In systemic autoimmune diseases, the immune system of an organism launches an immune response against the organism's own tissues, causing inflammation and tissue damage. Examples of diseases caused by immune dysfunction include rheumatoid arthritis, systemic lupus erythematosus, multiple sclerosis, scleroderma, psoriasis, and Sjorgen's syndrome. Additionally, there are other B cell related diseases in which immunoglobulin expression may play a role, such as multiple myeloma.

Relatively little is known about the role of light chain variable region expression in autoimmune diseases or other B cell related diseases. Thus, there is a need in the art for improved understanding of the relationship between immunoglobulin expression and disease.

### SUMMARY

The present invention provides a microarray comprising a plurality of oligonucleotide species at least 20 nucleotides long and capable of hybridizing to a polynucleotide comprising a sequence that encodes at least a portion of a light chain variable (LCV) region, or a complement thereof.

Also provided is a method of characterizing the light chain variable gene expression in a subject. First, B cells are isolated from the subject and target polynucleotides are prepared from the B cells. Then target polynucleotides are hybridized to a microarray of the invention. Finally, light chain variable gene expression is characterized by detecting hybridization of the target polynucleotides to one or more oligonucleotide species.

In another aspect, the invention provides methods of identifying light chain variable genes associated with a disease by comparing the light chain variable gene expression in a first subject with the disease to the light chain variable gene expression in a second subject that does not have the disease. The light chain variable gene expression can be assessed using a microarray according to the present invention. A difference in light chain variable gene expression between the first and the second subject indicating that expression of the light chain variable gene is associated with the disease.

In still another aspect, methods of monitoring a disease state in a subject are provided. The expression in the subject of a light chain variable gene associated with the disease is compared at two or more time points.

In a further aspect, methods of evaluating the effect of a therapy or a therapeutic agent on expression of a light chain variable gene associated with a disease in a subject are provided. The expression of the light chain variable gene in the subject is compared before and after treatment.

In a still further aspect, kits comprising the microarray are provided.

### BRIEF DESCRIPTION OF THE DRAWINGS

FIG. 1 is a graph showing fluorescence intensity of labeled human B3 light chain DNA after hybridization to the human light chain variable genes.

FIG. 2 is a graph demonstrating the light chain variable gene repertoire differences in B6 56R transgenic mice as compared to Balb/c mice. The microarray fluorescent intensity data was normalized to the kappa constant region for each sample and then the Balb/c normalized values were subtracted from the B6 56R transgenic normalized values. A positive number indicates an over-representation of the light chain variable region in the B6 56R transgenic mouse.

FIG. 3 is a graph demonstrating the light chain variable gene repertoire differences in B6 56R transgenic mice before and after induction of autoimmunity. The microarray fluorescent intensity data were normalized to the kappa constant region for each sample.

FIG. 4 is a graph demonstrating the light chain variable expression in B cells harvested from the cerebral spinal fluid of a patient with multiple sclerosis. The data are normalized to the kappa constant region.

FIG. 5 is a graph demonstrating the overrepresentation of light chain variable genes in B cells isolated from the cerebral spinal fluid of an individual with multiple sclerosis normalized to light chain variable expression in B cells isolated from the cerebral spinal fluid of three individuals not suffering from an autoimmune disease.

FIG. 6 is a graph comparing the peripheral B cell repertoires of a healthy individual and a systemic lupus erythematosus (SLE) patient. The x-axis is the sum of the intensities for a given light chain gene in both samples. The y-axis is the ratio (SLE:healthy) of intensities between the samples. Global intensity dependent normalization was performed for each sample.

### DETAILED DESCRIPTION

As described in detail below, the light chain variable gene repertoire expressed by an individual may provide information concerning that individual's risk of developing a disease, his prognosis, or his response to a particular treatment. A light chain variable gene associated with a disease can be determined by comparing expression of light chain variable genes of individuals with the disease to the expression of light chain variable genes of individuals who do not have the disease, and identifying genes that are differentially expressed among a subpopulation of individuals with the disease.

The present invention provides a new approach to evaluating autoimmune disease using microarray analysis of light chain variable (V) gene usage. Microarrays suitable for use in this analysis include oligonucleotide species capable of hybridizing to a polynucleotide encoding at least a portion of an antibody light chain variable region, or to a complement thereof. Microarray analysis provides a rapid and relatively inexpensive method of characterizing light chain variable gene expression in a subject. By this method, the light chain variable repertoire of a subject can be determined. Information can be obtained by comparing light chain variable gene repertoires between subjects or by evaluating changes in expression in a single subject over time.

The light chain variable region repertoire of a subject refers to the light chain variable genes expressed by a subject. Characterization of the light chain variable gene repertoire includes, but is not limited to, detection and/or quantification of one or more of the light chain variable genes expressed in a subject. The subject can be any subject capable of expressing light chain variable genes, e.g. vertebrates. In the Examples, mouse and human subjects were used.

The methods of the invention can identify and distinguish light chain variable gene repertoires and provide information on relative expression of individual light chain variable genes in both humans and mice. This information is useful in identifying those light chain variable genes associated with a disease, such as autoimmune diseases or other B cell related diseases. Once a light chain variable gene is identified as being associated with a particular disease, the expression of that light chain variable gene can be used to diagnose the disease and to predict or assess the course of disease (e.g., severity, flares, or remission). The microarray may also be used to evaluate the effect of a therapy or therapeutic agent on expression of light chain variable genes associated with disease, or to predict an individual's response to treatment. Light chain variable gene expression may also be used to predict auto-antibody structures or susceptibility to autoimmune disease. Microarrays according to the invention may also allow evaluation of the overall immune system function and/or status of a subject.

Mouse models of systemic autoimmune diseases described below demonstrate that certain light chain V genes have unique properties and the expression of certain sequences are associated with disease activity. Table 1 includes the light chain V regions of anti-DNA antibodies isolated from a mouse model of lupus. As can be seen in Table 1, these light chain variable regions have an unusually high frequency of acidic amino acids clustered in the complementarity determining regions (CDRs). Expression of these light chains in a subject with an autoimmune disease were studied using microarrays. Importantly, human counterparts to the mouse light chain variable genes discussed above have been identified, and were found to have similar structural features to those of mice (See Table 1, bottom panel). These light chains may also be important in autoimmune pathology, susceptibility and disease course in humans.

The human B2 gene encodes a κIII domain that displays four aspartic acids in a five amino acid segment of CDR1 (Table 1) and is thus of particular interest. A database of almost 300 human kappa light chain variable domain sequences derived from patients with monoclonal dyscrasias reveals no example of such an aspartic acid cluster, although studies suggest that B2 products are functional. One of the aspartic acids in B2 is located at amino acid position 31, which has been linked to the formation of amyloid fibrils in approximately 10% of patients with multiple myeloma. Specifically, mutations that generated an aspartic acid at position 31 are highly correlated with amyloid formation. Thus, light chain variable gene expression may also be related to symptomology or sequelae of a disease such as multiple myeloma.

Subsets of light chain variable regions have been found to be associated with particular diseases as described in detail below. Briefly, auto-antibodies found in individuals with systemic autoimmune diseases such as lupus, rheumatoid arthritis and multiple sclerosis (MS) have a restricted light chain repertoire. These auto-antibodies appear to be associated with pathogenesis and/or correlate with disease activity in systemic autoimmune diseases. Knowledge of antibodies associated with pathogenesis will yield important information concerning the structure and expression patterns of these light chain variable genes. Light chain variable gene expression is also relevant to other diseases such as multiple myeloma and other B cell-related diseases.

The development of a rapid, sensitive, reliable and relatively inexpensive means of analyzing immunoglobulin light chain expression in subjects is needed to determine whether particular light chain variable genes are related to a particular disease. Development of such methods will allow for improved diagnosis of these diseases and may aid in determining disease prognosis and etiology, monitoring disease progression and evaluating therapeutic agents and treatment regimens.

TABLE 1

Comparison of mouse light chain V genes (top) identified in lupus models as
having unique properties and human light chains (bottom) with similar properties.
Highlighted are the relevant residues that confer autoreactivity.

| | | |
|---|---|---|
| BT20 | STTVTQSPASLSMAIGEKVTIRCITSTDIDD----DMNWYQQKPGEPPKLLISE---- | SEQ ID NO: 1 |
| | GNTLRPGVPSRFSSSGYGTDFVFTIENMLSEDVADYYCLQSDNLP---- | |
| BW20 | ETTVTQSPASLSVATGEKVTIRCITSTDIDD----DMNWYQQKPGEPPKLLISE---- | SEQ ID NO: 2 |
| | GNTLRPGVPSRFSSSGYGTDFVFTIENTLSEDVADYYCLQSDNMP---- | |
| GJ39C | DIQMTQSPSSLSASLGGKVTITCKASQDINK----YIAWYQHKPGKGPRLLIHY---- | SEQ ID NO: 3 |
| | TSTLQPGIPSRFSGSGSGRDYSFSISNLEPEDIATYYCLQYDNLL---- | |
| VLX | QLVLTQ-SSSASFSLGASAKLTCTLSSQHST--- | SEQ ID NO: 4 |
| YTIEWYQQQPLKPPKYVMELKKDGSHSTGDGIPDRFSGSSSGADRYLSISNIQPEDEAIYIC | | |
| | GVGDTIKEQFV | |
| 21-4 | DIVLTQSPASLAVSLGQRATISCKASQSVDYDGDSYMNWYQQKPGQPPKILIYA---- | SEQ ID NO: 5 |
| | ASNLESGIPARFSGSGSGTDFTLNIHPVEEEDAATYYCQQSNEDP---- | |
| 12-38 | DIQMTQSPASLAASVGETVTITCRASENIYY----SLAWYQQKQGKSPQLLIYN---- | SEQ ID NO: 6 |
| | ANSLEDGVPSRFSGSGSGTQYSMKINSMQPEDTATYFCKQAYDVP---- | |
| 12-46 | DIQMTQSPASLSVSVGETVTITCRASENIYS----NLAWYQQKQGKSPQLLVYA---- | SEQ ID NO: 7 |
| | ATNLADGVPSRFSGSGSGTQYSLKINSLQSEDFGSYYCQHFWGTP---- | |
| 08/018 | DIQMTQSPSSLSASVGDRVTITCQASQDISN--YLNWYQQKYGKAPKLLIYDASN---- | SEQ ID NO: 8 |
| | LETGVPSRFSGSG-SGTDFTFTISSLQPEDIATYYCQQYDNLP---- | |
| L25 | EIVMTQSPATLSLSPGERATLSCRASQSVSSSY-LSWYQQKPGQAPRLLIYGAST---- | SEQ ID NO: 9 |
| | RATGIPARFSGSG-SGTDFTLTISSLQPEDFAVYYCQQDYNLP---- | |
| B2 | ETTLTQSPAFMSATPGDKVNISCKASQDIDD--DMNWYQQKPGEAAIFIIQEATT---- | SEQ ID NO: 10 |
| | LVPGIPPRFSGSG-YGTDFTLTINNIESEDAAYYFCLQHDNFP---- | |
| L11 | AIQMTQSPSSLSASVGDRVTITCRASQGIRN--DLGWYQQKPGKAPKLLIYAASS---- | SEQ ID NO: 11 |
| | LQSGVPSRFSGSG-SGTDFTLTISSLQPEDFATYYCLQDYNYP---- | |
| L10 | EIVMTQSPPTLSLSPGERVTLSCRASQSVSSSY-LTWYQQKPGQAPRLLIYGAST---- | SEQ ID NO: 12 |
| | RATSIPARESGSG-SGTDFTLTISSLQPEDFAVYYCQQDHNLPP--- | |
| V2-19 | SYELTQPSSVSVSPGQTARITCSGDVLAKKY---ARWFQQKYGQAPVLVIYKDSE---- | SEQ ID NO: 13 |
| | RPSGIPERFSGSS-SGTTVTLTISGAQVEDEADYYCYSAADNNL--- | |
| V2-15 | SYELTQLPSVSVSPGQTARITCSGDVLGENY---ADWYQQKPGQAPELVIYEDSE---- | SEQ ID NO: 14 |
| | RYPGIPERFSGST-SGNTTTLTISRVLTEDEADYYCLSGDEDNP--- | |
| V5-4 | QPVLTQSSSASASLGSSVKLTCTLSSGHSS-- | SEQ ID NO: 15 |
| | YIIAWHQQQPGKAPRYLMKLEGSGSYNKGSGVPDRFSGS- | |
| | SSGADRYLTISNLQFEDEADYYCETWDSNT---- | |

The microarray of the present invention includes a plurality of oligonucleotide species capable of hybridizing to a polynucleotide comprising a sequence encoding at least a portion of an antibody light chain variable region, or a complement thereof. As described in the Examples below, a bioinformatics approach was used to select oligonucleotide sequences for use in the microarray from the variable regions of the 99 mouse and 82 human light chain variable genes. The oligonucleotide sequences were selected to minimize cross-hybridization with each of the other light chain variable genes. Generally, the oligonucleotide sequences selected were between 60 and 80 nucleotides long. Computer programs suitable for use in the selection process are described in detail in the Examples section. However, as one skilled in the art will appreciate, any suitable program for selecting oligonucleotide sequences can be used, and many different programs are known to those of skill in the art. The light chain variable regions from all species identified to date are similar in structure such that one of skill in the art would expect the microarray and methods described herein could be adapted for use in any species capable of producing antibodies.

The human and mouse light chain variable gene specific oligonucleotides listed in Table 2 and Table 3, respectively, were selected from the germline sequences for the genes based on several criteria. The oligonucleotides were chosen from the most variable regions of each light chain variable gene, and were selected to be sufficiently unique to allow identification of individual light chain variable regions with minimal cross-hybridization. The oligonucleotides were also selected to maximize the likelihood that all of the sequences would hybridize to their target sequences under similar conditions by choosing a group of oligonucleotides that have similar G-C content and similar melting temperatures. Finally, oligonucleotides that have a low potential to self-fold were selected. Any suitable criteria could be used to select oligonucleotides for use in the microarray. Additional potential oligonucleotides are listed in Tables 4 and 5.

One of skill in the art will appreciate that the present invention is not limited to the oligonucleotides listed in Tables 2-5. Additional oligonucleotides for use in the microarray and methods of the invention include, but are not limited to, the complements of the oligonucleotides listed in the Tables, oligonucleotides substantially similar to the oligonucleotides listed in the Tables and any other oligonucleotides derived from the germline sequences of the light chain variable regions. The light chain variable region gene sequences are publicly available in GenBank under the heading “Ig Germline Genes”. “Substantially similar oligonucleotides” includes oligonucleotides with at least 90% nucleotide identity to the oligonucleotides of Table 2-5. Suitably the oligonucleotides have at least 95% nucleotide identity to the oligonucleotides of Tables 2-5. Also included are light chain variable oligonucleotides containing portions of the sequences of the oligonucleotides listed in Tables 2-5.

In the Examples, oligonucleotides between 60 and 80 nucleotides long were used to minimize cross-hybridization with multiple light chain variable regions. One of skill in the art would appreciate that shorter or longer oligonucleotides could be used. Use of shorter oligonucleotides may result in a loss of specificity for a single light chain variable region, but such a loss of specificity can be compensated for by selecting and using multiple shorter oligonucleotides for each light chain variable region and then using a computer program that compensates for the cross-hybridization in the analysis of the microarray data. For example, the oligonucleotides included in the microarray may suitably be at least 20 nucleotides long, 30 nucleotides long, 40 nucleotides long, 50 nucleotides long, 60 nucleotides long, 70 nucleotides long, 80 nucleotides long, or 100 nucleotides long. Quantification of cross-hybridization between light chain variable region oligonucleotides and all target polynucleotides can be tested using target polynucleotides complementary to each of the oligonucleotide species on the microarray. These target polynucleotides may be synthetically produced or produced from B cell clones expressing known light chain variable regions. The results from such cross-hybridization experiments can then be applied to experimental data to eliminate experimental artifacts due to cross-hybridization. Other methods to minimize or compensate for cross-hybridization may also be used as would be apparent to those of skill in the art.

TABLE 2

Human light chain V gene selected oligonucleotides. The complements of these
sequences may also be used.

| | |
|---|---|
| Gene | Sequence |

| | | |
|---|---|---|
| A17/A1 | CTGCAGGTCTAGTCAAAGCCTCGTATACAGTGATGGAAACACCTACTTGAATTGG | SEQ ID NO: 16 |
| | TTTCAGCAGAGG | |
| A10 | CTCACCATCAATAGCCTGGAAGCTGAAGATGCTGCAACGTATTACTGTCATCAGA | SEQ ID NO: 17 |
| | GTAGTAGTTTAC | |
| A27/A11 | GACTTCACTCTCACCATCAGCAGACTGGAGCCTGAAGATTTTGCAGTGTATTACT | SEQ ID NO: 18 |
| | GTCAGCAGTATG | |
| A14 | GATCTGGGACAGATTTCACCTTTACCATCAGTAGCCTGGAAGCTGAAGATGCTGC | SEQ ID NO: 19 |
| | AACATATTACTG | |
| A2/A18b | CATCTCCTGCAAGTCTAGTCAGAGCCTCCTGCATAGTGATGGAAAGACCTATTTG | SEQ ID NO: 20 |
| | TATTGGTACCTG | |
| A3/A19 | GATCAGGCACAGATTTTACACTGAAAATCAGCAGAGTGGAGGCTGAGGATGTTG | SEQ ID NO: 21 |
| | GGGTTTATTACTG | |
| L14/A20 | TCTGCATCTGTAGGAGACAGAGTCACCATCACTTGCCGGGCGAGTCAGGGCATT | SEQ ID NO: 22 |
| | AGCAATTATTTAG | |
| A23 | CTACTTGAGTTGGCTTCAGCAGAGGCGAGGCCAGCCTCCAAGACTGCTAATTTAT | SEQ ID NO: 23 |
| | AAGATTTCTAAC | |
| A30 | CTCTCACAATCAGCAGCCTGCAGCCTGAAGATTTTGCAACTTATTACTGTCTACA | SEQ ID NO: 24 |
| | GCATAATAGTTA | |
| A7 | CATCTCCTTCAGGTCTAGTCAAAGCCTCGTACACAGTGATGGAAACACCTACTTG | SEQ ID NO: 25 |
| | AGTTGGCTTCAG | |
| B2 | GAGAAGCTGCTATTTTCATTATTCAAGAAGCTACTAGTCTCGTTCCTGGAATCCC | SEQ ID NO: 26 |
| | ACCTCGATTCAG | |
| B3 | CATCAACTGCAAGTCCAGCCAGAGTGTTTTATACAGCTCCAACAATAAGAACTA | SEQ ID NO: 27 |
| | CTTAGCTTGGTAC | |
| L1 | GTAGGAGACAGAGTCACCATCACTTGTCGGGCGAGTCAGGGCATTAGCAATTAT | SEQ ID NO: 28 |
| | TTAGCCTGGTTTC | |
| L10 | CTCACCATCAGCAGCCTGCAGCCTGAAGATTTTGCAGTTTATTACTGTCAGGAGG | SEQ ID NO: 29 |
| | ATCATAACTTAC | |
| L11 | CTCTCACCATCAGCAGCCTGCAGCCTGAAGATTTTGCAACTTATTACTGTCTACA | SEQ ID NO: 30 |
| | AGATTACAATTA | |
| L12 | TGGTATCAGCAGAAACCAGGGAAAGCCCCTAAGCTCCTGATCTATGATGCCTCC | SEQ ID NO: 31 |
| | AGTTTGGAAAGTG | |
| L15 | CTCTCACCATCAGCAGCCTGCAGCCTGAAGATTTTGCAACTTATTACTGCCAACA | SEQ ID NO: 32 |
| | GTATAATAGTTA | |
| L2/L16 | CTCTCACCATCAGCAGCCTGCAGTCTGAAGATTTTGCAGTTTATTACTGTCAGCA | SEQ ID NO: 33 |
| | GTATAATAACTG | |
| L14/18a/L18 | TGGTATCAGCAGAAACCAGGGAAAGCTCCTAAGCTCCTGATCTATGATGCCTCC | SEQ ID NO: 34 |
| | AGTTTGGAAAGTG | |
| L19 | CATCTTCTGTGTCTGCATCTGTAGGAGACAGAGTCACCATCACTTGTCGGGCGAG | SEQ ID NO: 35 |
| | TCAGGGTATTAG | |
| L6/L20 | TTCACTCTCACCATCAGCAGCCTAGAGCCTGAAGATTTTGCAGTTTATTACTGTC | SEQ ID NO: 36 |
| | AGCAGCGTAGCA | |
| L22 | GTAGGAGACAGAGTCAGTATCATTTGCTGGGCAAGTGAGGGCATTAGCAGTAAT | SEQ ID NO: 37 |
| | TTAGCCTGGTATC | |
| L23 | CTCTCACCATCAGCAGCCTGCAGCCTGAAGATTTTGCAACTTATTACTGTCAACA | SEQ ID NO: 38 |
| | GTATTATAGTAC | |
| L24 | CATCTACAGGAGACAGAGTCACCATCAGTTGTCGGATGAGTCAGGGCATTAGCA | SEQ ID NO: 39 |
| | GTTATTTAGCCTG | |
| L25 | CTCACCATCAGCAGCCTGCAGCCTGAAGATTTTGCAGTTTATTACTGTCAGCAGG | SEQ ID NO: 40 |
| | ATTATAACTTAC | |
| L5 | GACAGATTTCACTCTCACCATCAGCAGCCTGCAGCCTGAAGATTTTGCAACTTAC | SEQ ID NO: 41 |
| | TATTGTCAACAG | |
| L8 | CTCTCACAATCAGCAGCCTGCAGCCTGAAGATTTTGCAACTTATTACTGTCAACA | SEQ ID NO: 42 |
| | GCTTAATAGTTA | |
| L9 | CATCTACAGGAGACAGAGTCACCATCACTTGTCGGGCGAGTCAGGGTATTAGCA | SEQ ID NO: 43 |
| | GTTATTTAGCCTG | |
| O11/O1-72 | GTCTAGTCAGAGCCTCTTGGATAGTGATGATGGAAACACCTATTTGGACTGGTAC | SEQ ID NO: 44 |
| | CTGCAGAAGCCA | |
| O2/O12 | GAGCATTAGCAGCTATTTAAATTGGTATCAGCAGAAACCAGGGAAAGCCCCTAA | SEQ ID NO: 45 |
| | GCTCCTGATCTAT | |
| O4/O14 | CAGTTATTTAAATTGGTATCGGCAGAAACCAGGGAAAGTTCCTAAGCTCCTGATC | SEQ ID NO: 46 |
| | TATAGTGCATCC | |
| O8/O18 | GTAGGAGACAGAGTCACCATCACTTGCCAGGCGAGTCAGGACATTAGCAACTAT | SEQ ID NO: 47 |
| | TTAAATTGGTATC | |
| V1-11 | GCAGAGGGTCACCATCTCCTGTTCTGGAAGCAGCTCCAACATCGGAAATAATGC | SEQ ID NO: 48 |
| | TGTAAACTGGTAC | |
| V1-16 | GCAGAGGGTCACCATCTCTTGTTCTGGAAGCAGCTCCAACATCGGAAGTAATACT | SEQ ID NO: 49 |
| | GTAAACTGGTAC | |
| V1-17 | GCAGAGGGTCACCATCTCTTGTTCTGGAAGCAGCTCCAACATCGGAAGTAATTAT | SEQ ID NO: 50 |
| | GTATACTGGTAC | |
| V1-18 | CTCCCTGGCCATCACTGGACTCCAGTCTGAGGATGAGGCTGATTATTACTGCAAA | SEQ ID NO: 51 |
| | GCATGGGATAAC | |
| V1-19 | CAAACTCCTCATTTATGACAATAATAAGCGACCCTCAGGGATTCCTGACCGATTC | SEQ ID NO: 52 |
| | TCTGGCTCCAAG | |
| V1-2 | GTCAGTCACCATCTCCTGCACTGGAACCAGCAGTGACGTTGGTGGTTATAACTAT | SEQ ID NO: 53 |
| | GTCTCCTGGTAC | |
| V1-20 | CCACCCTCCCAAACTCCTATCCTACAGGAATAACAACCGGCCCTCAGGGATCTCA | SEQ ID NO: 54 |
| | GAGAGATTCTCT | |
| V1-22 | CTGTGATCTATGAGGATAACCAAAGACCCTCTGGGGTCCCTGATCGGTTCTCTGG | SEQ ID NO: 55 |
| | CTCCATCGACAG | |
| V1-3 | GTCAGTCACCATCTCCTGCACTGGAACCAGCAGTGATGTTGGTGGTTATAACTAT | SEQ ID NO: 56 |
| | GTCTCCTGGTAC | |
| V1-4 | CAAACTCATGATTTATGAGGTCAGTAATCGGCCCTCAGGGGTTTCTAATCGCTTC | SEQ ID NO: 57 |
| | TCTGGCTCCAAG | |
| V1-5 | CTCTGGGCTCCAGGCTGAGGACGAGGCTGATTATTACTGCAGCTTATATACAAGC | SEQ ID NO: 58 |
| | AGCAGCACTTTC | |
| V1-7 | CTGACAATCTCTGGGCTCCAGGCTGAGGACGAGGCTGATTATTACTGCTGCTCAT | SEQ ID NO: 59 |
| | ATGCAGGTAGTAG | |
| V1-9 | CTCTGGGCTCAAGTCCGAGGTTGAGGCTAATTATCACTGCAGCTTATATTCAAGT | SEQ ID NO: 60 |
| | AGTTACACTTTC | |
| V2-1 | GATAAATATGCTTGCTGGTATCAGCAGAAGCCAGGCCAGTCCCCTGTGCTGGTCA | SEQ ID NO: 61 |
| | TCTATCAAGATAG | |
| V2-11 | TAGTCACATTGACCATCAGTGGAGTCCAGGCAGAAGACGAGGCTGACTATTACT | SEQ ID NO: 62 |
| | GTCTATCAGCAGA | |
| V2-13 | CTTGGGACAGACAGTCAGGATCACATGCCAAGGAGACAGCCTCAGAAGCTATTA | SEQ ID NO: 63 |
| | TGCAAGCTGGTAC | |
| V2-15 | CTGAGTTGGTGATATACGAAGATAGTGAGCGGTACCCTGGAATCCCTGAACGAT | SEQ ID NO: 64 |
| | TCTCTGGGTCCAC | |
| V2-17 | GACAACAGTCACGTTGACCATCAGTGGAGTCCAGGCAGAAGATGAGGCTGACTA | SEQ ID NO: 65 |
| | TTACTGTCAATCAG | |
| V2-6 | CTGGGACAGACGGCCAGGATTACCTGTGGGGGAAACAACATTGGAAGTAAAAAT | SEQ ID NO: 66 |
| | GTGCACTGGTACC | |
| V3-2 | GTTACTATCCAAACTGGTTCCAGCAGAAACCTGGACAAGCACCCAGGGCACTGA | SEQ ID NO: 67 |
| | TTTATAGTACAAG | |
| V3-3 | GACACTGATTTATGATAGAAGCAACAAACACTCCTGGACACCTGCCCGGTTCTCA | SEQ ID NO: 68 |
| | GGCTCCCTCCTT | |
| V3-4 | CTGGAGGGACAGTCACACTCACTTGTGGCTTGAGCTCTGGCTCAGTCTCTACTAG | SEQ ID NO: 69 |
| | TTACTACCCCAG | |
| V4-1 | AATACAGGGATTTTACTCATCTCCGGGCTCCAGTCTGAGGATGAGGCTGACTATT | SEQ ID NO: 70 |
| | ACTGTATGATTT | |
| V4-2 | AATGCAGGGATTTTACTCATCTCTGGGCTCCAGTCTGAGGATGAGGCTGACTATT | SEQ ID NO: 71 |
| | ACTGTATGATTT | |
| V4-3 | AATGCAGGGATTTTAGTCATCTCTGGGCTCCAGTCTGAGGATGAGGCTGACTATT | SEQ ID NO: 72 |
| | ACTGTATGATTT | |
| V4-4 | GGTACCAACAAAAGCCAGGGAACCCTCCCCGGTATCTCCTGTACTACCACTCAG | SEQ ID NO: 73 |
| | ACTCCAATAAGGG | |
| V4-6 | GTACCAGCAGAAGCCAGGGAGCTCTCCCAGGTTATTCCTGTATCACTACTCAGAC | SEQ ID NO: 74 |
| | TCAGACAAGCAG | |
| V5-1 | CATCGAATGGTATCAACAGAGACCAGGGAGGTCCCCCCAGTATATAATGAAGGT | SEQ ID NO: 75 |
| | TAAGAGTGATGGC | |
| V5-2 | GAATCGGTACCTGACCATCAAGAACATCCAGGAAGAAGATGAGAGTGACTACCA | SEQ ID NO: 76 |
| | CTGTGGGGCAGAC | |
| V5-4 | CTACCTCACCATCTCCAACCTCCAGTTTGAGGATGAGGCTGATTATTACTGTGAG | SEQ ID NO: 77 |
| | ACCTGGGACAGT | |
| V1-13_146 | TCATCTATGGTAACAGCAATCGGCCCTCAGGGGTCCCTGACCGATTCTCTGGCTC | SEQ ID NO: 78 |
| | CAAGTCTGGCACCTCA | |
| V2-14_127 | CCTGTGCTGGTCGTCTATGATGATAGCGACCGGCCCTCAGGGATCCCTGAGCGAT | SEQ ID NO: 79 |
| | TCTCTGGCTCCAACT | |
| V2-19_209 | TCACCTTGACCATCAGCGGGGCCCAGGTTGAGGATGAGGCTGACTATTACTGTTA | SEQ ID NO: 80 |
| | CTCTGCGGCTGACAACA | |
| V2-7_150 | CAGCAAACGACCCTCCGGGATCCCTGAGAGATTCTCTGGCTCCAGCTCAGGGAC | SEQ ID NO: 81 |
| | AATGGCCACCTTGACTATC | |
| V2-8_129 | TGTGCTGGTCATCTATAGCGATAGCAACCGGCCCTCAGGGATCCCTGAGCGATTC | SEQ ID NO: 82 |
| | TCTGGCTCCAACCCAG | |
| V5-6_121 | GAGAAGGGCCCTCGGTACTTGATGAAGCTTAACAGTGATGGCAGCCACAGCAAG | SEQ ID NO: 83 |
| | GGGGACGGGATCCCTGATC | |
| actin_beta | TTTTAATAGTCATTCCAAATATGAGATGCGTTGTTACAGGAAGTCCCTTGCCATC | SEQ ID NO: 84 |
| | CTAAAAGCCACC | |
| CD19 | GCTGTGACTTTGGCTTATGTGATCTTCTGCCTGTGTTCCCTTGTGGGCATTCTTCA | SEQ ID NO: 85 |
| | TCTTCAAAGAG | |
| CD20 | CAATACAGAACCCATTCCATTTATCTTTGTACAGGGCTGACATTGTGGCACATTC | SEQ ID NO: 86 |
| | TTAGAGTTACCA | |
| hk_con | GAATAACTTCTATCCCAGAGAGGCCAAAGTACAGTGGAAGGTGGATAACGCCCT | SEQ ID NO: 87 |
| | CCAATCGGGTAAC | |

TABLE 3

Mouse light chain V gene selected oligonucleotides. The complements of
these sequences may also be used.

| | |
|---|---|
| Gene | Sequence |

| | | |
|---|---|---|
| 12-38 | CTGGGACACAGTATTCTATGAAGATCAACAGCATGCAGCCTGAAGATACCGCA | SEQ ID NO: 88 |
| | ACTTATTTCTGTAAACA | |
| 12-41 | GCAAAAACCTTAGCAGATGGTGTGCCATCAAGGTTCAGTGGCAGTGGATCAGG | SEQ ID NO: 89 |
| | AACACAATATTCTCTCA | |
| 12-44 | AGTTTTCTCTGAAGATCAACAGCCTGCAGCCTGAAGATTTTGGGAGTTATTACT | SEQ ID NO: 90 |
| | GTCAACATCATTATGG | |
| 12-46 | GCAACAAACTTAGCAGATGGTGTGCCATCAAGGTTCAGTGGCAGTGGATCAGG | SEQ ID NO: 91 |
| | CACACAGTATTCCCTCA | |
| 19-13 | TTTCACTCTCACCATCAGCAATATGCAGTCTGAAGACCTGGCAGATTATTTCTG | SEQ ID NO: 92 |
| | CCAGCAATATAGCAGC | |
| 19-14 | CTCTCACCATTAGCAATGTGCAATCTGAAGACCTGGCAGATTATTTCTGTCTGC | SEQ ID NO: 93 |
| | AACATTGGAATTATCC | |
| 19-15 | GGTACTAATGTAGCCTGGTATCAACAGAAACCAGGGCAATCTCCTAAAGCACT | SEQ ID NO: 94 |
| | GATTTACTCGGCATCCT | |
| 19-17 | GGACGGATTTCACTTTCACCATCAGCAGTGTGCAGGCTGAAGACCTGGCAGTT | SEQ ID NO: 95 |
| | TATTACTGTCAGCAACA | |
| 19-20 | CACTGGGGTCCCCGATCGCTTCACAGGCAGTGGATCTGCAACAGATTTCACTCT | SEQ ID NO: 96 |
| | GACCATCAGCAGTGTG | |
| 19-23 | CTCTCACCATTAGCAATGTGCAGTCTGAAGACTTGGCAGATTATTTCTGTCAGC | SEQ ID NO: 97 |
| | AATATAGCAGCTATCC | |
| 19-25 | TATACTCTCACCATCAGCAGTGTGCAGGCTGAAGACCTGGCACTTTATTACTGT | SEQ ID NO: 98 |
| | CAGCAACATTATAGCA | |
| 19-29 | ACCCTGGGGTCCCTGATCGCTTCACAGGCAGTGGATCTGCAACAGATTTCACTC | SEQ ID NO: 99 |
| | TGACCATCAGCAGTCT | |
| 19-32 | ATTCCTGCTTGTATCAGCAGGAGACAGGGTTACCATAACCTGCAAGGCCAGTC | SEQ ID NO: 100 |
| | AGAGTGTGAGTAATGAT | |
| 21-1 | CACCATCTCCTGCAGAGCCAGTGAAAGTGTTGAATATTATGGCACAAGTTTAA | SEQ ID NO: 101 |
| | TGCAGTGGTACCAACAG | |
| 21-10 | TCACCCTCACCATTGATCCTGTGGAGGCTGATGATGCTGCAACCTATTACTGTC | SEQ ID NO: 102 |
| | AGCAAAATAATGAGGA | |
| 21-12 | CTGGCTATAGTTATATGCACTGGTACCAACAGAAACCAGGACAGCCACCCAAA | SEQ ID NO: 103 |
| | CTCCTCATCTATCTTGC | |
| 21-2 | GCCACCATCTCCTGCAGAGCCAGCGAAAGTGTTGATAATTATGGCATTAGTTTT | SEQ ID NO: 104 |
| | ATGAACTGGTTCCAAC | |
| 21-3 | GAGCCACTATCTTCTGCAGAGCCAGCCAGAGTGTCGATTATAATGGAATTAGT | SEQ ID NO: 105 |
| | TATATGCACTGGTTCCA | |
| 21-4 | GCCACCATCTCCTGCAAGGCCAGCCAAAGTGTTGATTATGATGGTGATAGTTAT | SEQ ID NO: 106 |
| | ATGAACTGGTACCAAC | |
| 21-5 | CTCACCATTAATCCTGTGGAGGCTGATGATGTTGCAACCTATTACTGTCAGCAA | SEQ ID NO: 107 |
| | AGTAATGAGGATCCTC | |
| 21-7 | CTAGCTATAGTTATATGCACTGGTACCAACAGAAACCAGGACAGCCACCCAAA | SEQ ID NO: 108 |
| | CTCCTCATCAAGTATGC | |
| 21-9 | GACAGAGGGCCACCATATCCTGCCAAGCCAGCGAAAGTGTCAGTTTTGCTGGT | SEQ ID NO: 109 |
| | ACAAGTTTAATGCACTG | |
| 22-33 | AGTAAGAAGGTCACCATTAGTTGCACGGCCAGTGAGAGCCTTTATTCAAGCAA | SEQ ID NO: 110 |
| | ACACAAGGTGCACTACT | |
| 23-37 | GATTACACTCTCAGTATCAACAGTGTGAAGCCCGAAGATGAAGGAATATATTA | SEQ ID NO: 111 |
| | CTGTCTTCAAGGTTACA | |
| 23-39 | CTCTCAGTATCAACAGTGTGGAACCTGAAGATGTTGGAGTGTATTACTGTCAA | SEQ ID NO: 112 |
| | AATGGTCACAGCTTTCC | |
| 23-43 | CAGGAGATAGCGTCAGTCTTTCCTGCAGGGCCAGCCAAAGTATTAGCAACAAC | SEQ ID NO: 113 |
| | CTACACTGGTATCAACA | |
| 23-45 | GGAGATAGAGTCAGTCTTTCCTGCAGGGCCAGTCAAAGTATTAGCAACTACCT | SEQ ID NO: 114 |
| | ACACTGGTATCAACAAA | |
| 23-48 | TTCTCCTGCAGGGCCAGTCAGAGCATTGGCACAAGCATACACTGGTATCAGCA | SEQ ID NO: 115 |
| | AAGAACAAATGGTTCTC | |
| 4-50 | GTACTGGTACCAGCAGAAGTCAGATGCCTCCCCCAAACTATGGATTTATTACA | SEQ ID NO: 116 |
| | CATCCAACCTGGCTCCT | |
| 4-51 | GGGGCTGGGATCTCTTACTCTCTCACAATCAGCAGCATGGAGGCTGAAAATGA | SEQ ID NO: 117 |
| | TGCAACTTATTACTGCC | |
| 4-57 | TGTCTGCATCTCCAGGGGAAAAGGTCACCATGACCTGCAGGGCCAGCTCAAGT | SEQ ID NO: 118 |
| | GTAAGTTCCAGTTACTT | |
| 8-16 | ATCAGAATCTTTTATGGAGTGGAAACCAAAGGTACTGTTTGGTCTGGCACCAG | SEQ ID NO: 119 |
| | TGGAAACCAGGGCAAAC | |
| 8-19 | CTCTCACCATCAGCAGTGTGCAGGCTGAAGACCTGGCAGTTTATTACTGTCAGA | SEQ ID NO: 120 |
| | ATGATTATAGTTATCC | |
| 8-21 | GGTCACTATGAGCTGCAAATCCAGTCAGAGTCTGCTCAACAGTAGAACCCGAA | SEQ ID NO: 121 |
| | AGAACTACTTGGCTTGG | |
| 8-24 | TCCTCCCTGGCTATGTCAGTAGGACAGAAGGTCACTATGAGCTGCAAGTCCAG | SEQ ID NO: 122 |
| | TCAGAGCCTTTTAAATA | |
| 8-27 | GAAAAGGTCACTATGAGCTGTAAGTCCAGTCAAAGTGTTTTATACAGTTCAAA | SEQ ID NO: 123 |
| | TCAGAAGAACTACTTGG | |
| 8-28 | CCGATTTCACTCTTACCATCAGCAGTGTGCAGGCTGAAGACCTGGCAGTTTATT | SEQ ID NO: 124 |
| | ACTGTCAGAATGATCA | |
| 8-30 | CCCTAGCTGTGTCAGTTGGAGAGAAGGTTACTATGAGCTGCAAGTCCAGTCAG | SEQ ID NO: 125 |
| | AGCCTTTTATATAGTAG | |
| 8-34 | TAGCTAGTGGCAACCAAAATAACTACTTGGCCTGGCACCAGCAGAAACCAGGA | SEQ ID NO: 126 |
| | CGATCTCCTAAAATGCT | |
| RF | GAGAAACCTGGGAAAACTAATAAGCTTCTTATCTACTCTGGATCCACTTTGCAA | SEQ ID NO: 127 |
| | TCTGGAATTCCATCAA | |
| VL1 | AACTATGCCAACTGGGTCCAAGAAAAAGCAGATCATTTATTCACTGGTCTAAT | SEQ ID NO: 128 |
| | AGGTGGTACCAACAACC | |
| VL2 | TAACTATGCCAACTGGGTTCAAGAAAAACCAGATCATTTATTCACTGGTCTAAT | SEQ ID NO: 129 |
| | AGGTGGTACCAGCAAC | |
| VLx | ACAGCCACTCAAGCCTCCTAAGTATGTGATGGAGCTTAAGAAAGATGGAAGCC | SEQ ID NO: 130 |
| | ACAGCACAGGTGATGGG | |
| aa4 | CTCAAGTGTAAGTTACATGTACTGGTACCAGCAGAAGCCAGGATCCTCCCCCA | SEQ ID NO: 131 |
| | AACCCTGGATTTATCGC | |
| ac4 | TCACGATCAGCAGCATGGAGGCTGAAGATGTTGCCACTTATTACTGTTTTCAGG | SEQ ID NO: 132 |
| | GGAGTGGGTACCCACT | |
| ad4 | TATTCTCTCACAATCAGCAGCATGGAGGCTGAAGATGCTGCCACTTTTTACTGC | SEQ ID NO: 133 |
| | CAGCAGTACAGTGGTT | |
| ae4 | ACTCTCTCACAATCAGCAGCATGGAGGCTGAAGATGCTGCCTCTTATTTCTGCC | SEQ ID NO: 134 |
| | ATCAGTGGAGTAGTTA | |
| af4 | CAATCATGTCTGCATCTCTAGGGGAGGAGATCACCCTAACCTGCAGTGCCAGC | SEQ ID NO: 135 |
| | TCGAGTGTAAGTTACAT | |
| ag4 | CACTTCTACCAAATTCTGGATTTATAGGACATCCAACCTGGCTTCAGAAGTCCC | SEQ ID NO: 136 |
| | AGCTCCCTTCAGTGGC | |
| ah4 | TACTTGTACTGGTACCAGCAGAAGTCAGGATCCTCCCCAAAACTCTGGATTTAT | SEQ ID NO: 137 |
| | AGCATATCCAACCTGG | |
| ai4 | CTCTCACAATCAGCAGCATGGAGGCTGAAGATGCTGCCACTTATTACTGCCAC | SEQ ID NO: 138 |
| | CAGTATCATCGTTCCCC | |
| aj4 | TACATGTAATGGTTCCAGCAGAAGCGAGGATCCTCCCCCAAACTCTGGATTTAT | SEQ ID NO: 139 |
| | AGCATATCCAACCTGG | |
| al4 | CATGCACTGGTACCAGCAGAAGCCAGGATCCTCCCCCAGACTCTGGATTTATTT | SEQ ID NO: 140 |
| | AACATTCAACTTGGCT | |
| am4 | TTGTTCTCTCCCAGTCTCCAGCAATCCTGTCTGCATCTCCAGGGGAGAAGGTCA | SEQ ID NO: 141 |
| | CAATGACTTGCAGGGC | |
| an4 | GACATCTTTCTCTTTCACAATCAACAGCATGGAGGCTGAAGATGTTGCCACTTA | SEQ ID NO: 142 |
| | TTACTGTCAGCAAAGG | |
| ap4 | GCCAGCTCAAGTGTAAGTTACATGCACTGGTTCCAGCAGAAGCCAGGCACTTC | SEQ ID NO: 143 |
| | TCCCAAACTCTGGATTT | |
| aq4 | GTAAGTTACATGTACTGGTACCAGCAGAAGCCAAGATCCTCCCCCAAACCCTG | SEQ ID NO: 144 |
| | GATTTATCTCACATCCA | |
| ar4 | TGTAAGTTACATGTACAGGTACCAGCAGAAGCCAGGATCCTCACCCAAACCCT | SEQ ID NO: 145 |
| | GGATTTATGGCACATCC | |
| at4 | GTAAGTTACATGTACTGGTACCAGCAGAAGCCAGGATCCTCCCCCAGACTCCT | SEQ ID NO: 146 |
| | GATTTATGACACATCCA | |
| ay4 | GGTCTGAGAGCTCTTACACTCTGACAATCAGCTGCATGCAGGACGAAGTTGCT | SEQ ID NO: 147 |
| | GCCACTTACTATTGTCA | |
| ba4 | CCATGTATGCATCTCTAGGAGAGAGAGTCACTATCACTTGCAAGGCGAGTCAG | SEQ ID NO: 148 |
| | GACATTAATAGCTATTT | |
| bb1 | CACCTATTTACATTGGTACCTGCAGAAGCCAGGCCAGTCTCCAAAGCTCCTGAT | SEQ ID NO: 149 |
| | CTACAAAGTTTCCAAC | |
| bb1.1 | AATGGAAACACCTATTTATATTGGTACCTGCAGAAGCCAGGCCAGTCTCCAAA | SEQ ID NO: 150 |
| | GCTCCTGATCTACAGGG | |
| bd2 | GTCAGAGCCTCTTAGATAGTGATGGAAAGACATATTTGAATTGGTTGTTACAG | SEQ ID NO: 151 |
| | AGGCCAGGCCAGTCTCC | |
| bi2 | TTACAACAGAGGCCTGGCCAGGCTCCAAAGCACCTAATGTATCAGGTGTCCAA | SEQ ID NO: 152 |
| | ACTGGACCCTGGCATCC | |
| bj2 | ATATAGTAATGGAAAAACCTATTTGAATTGGTTATTACAGAGGCCAGGCCAGT | SEQ ID NO: 153 |
| | CTCCAAAGCGCCTAATC | |
| bl1 | CAGGTCTAGTCAGAGCCTTGAAAACAGTAATGGAAACACCTATTTGAACTGGT | SEQ ID NO: 154 |
| | ACCTCCAGAAACCAGGC | |
| bt20 | CATCCCTGTCCATGGCTATAGGAGAAAAAGTCACCATCAGATGCATAACCAGC | SEQ ID NO: 155 |
| | ACTGATATTGATGATGA | |
| bv9 | ACATTGGTAGTAGCTTAAACTGGCTTCAGCAGGAACCAGATGGAACTATTAAA | SEQ ID NO: 156 |
| | CGCCTGATCTACGCCAC | |
| bw20 | CCTGTCCGTGGCTACAGGAGAAAAAGTCACTATCAGATGCATAACCAGCACTG | SEQ ID NO: 157 |
| | ATATTGATGATGATATG | |
| cb9 | GGGAGACAGAATAACCATCACTTGCCAGGCAACTCAAGACATTGTTAAGAATT | SEQ ID NO: 158 |
| | TAAACTGGTATCAGCAG | |
| ce9 | CTCTCACCATTAGCAACCTGGAGCAAGAAGATATTGCCACTTACTTTTGCCAAC | SEQ ID NO: 159 |
| | AGGGTAATACGCTTCC | |
| cf9 | TTGCAGCAGAAACCAGGGAAATCATTTAAGGGCCTGATCTATCATGGAACCAA | SEQ ID NO: 160 |
| | CTTGGAAGATGGAGTTC | |
| ci12 | TCTGGGAGAAAGTGTCACCATCACATGCCTGGCAAGTCAGACCATTGGTACAT | SEQ ID NO: 161 |
| | GGTTAGCATGGTATCAG | |
| cj9 | CCGGGCAAGTCAGGACATTCATGGTTATTTAAACTTGTTTCAGCAGAAACCAG | SEQ ID NO: 162 |
| | GTGAAACTATTAAACAC | |
| cp9 | TTCTCTCACCATCAGCAACCTGGAACCTGAAGATATTGCCACTTACTATTGTCA | SEQ ID NO: 163 |
| | GCAGTATAGTAAGCTT | |
| cr1 | CTAGTCAGAGCATTGTACATAGTAATGGAAACACCTATTTAGAATGGTACCTG | SEQ ID NO: 164 |
| | CAGAAACCAGGCCAGTC | |
| cs1 | CACTCAAGATCAGCACAATAAAGCCTGAGGACTTGGGAATGTATTACTGCTTA | SEQ ID NO: 165 |
| | CAAGGTACACATCAGCC | |
| cv1 | AGATCAAGCCTCTATCTCTTGCAAGTCTACTAAGAGTCTTCTGAATAGTGATGG | SEQ ID NO: 166 |
| | ATTCACTTATTTGGAC | |
| cw9 | AAATTAGTGGTTACTTAAGCTGGCTTCAGCAGAAACCAGATGGAACTATTAAA | SEQ ID NO: 167 |
| | CGCCTGATCTACGCCGC | |
| cy9 | CAGTCTCTCTTGTCGGGCTAGTCAGGGCATTAGAGGTAATTTAGACTGGTATCA | SEQ ID NO: 168 |
| | GCAGAAACCAGGTGGA | |
| dv-36 | GCAGAAAGCAGAGCAAGTTCCCCGGCTCCTTATCCATAGTGCCTCCACTAGGG | SEQ ID NO: 169 |
| | CCGGTGGTGTCCCAGTC | |
| fl12 | AGTATTCTCTCAAGATCAGTAGCCTGCATCCTGACGATGTTGCAACGTATTACT | SEQ ID NO: 170 |
| | GTCAAAATGTGTTAAG | |
| gj38c | ACCAACACAAGCCTGGAAAAGGTCCTAGGCTGCTCATACATTACACATCTACA | SEQ ID NO: 171 |
| | TTACAGCCAGGCATCCC | |
| gm33 | CTCCTTTCTGTATCTCTAGGAGACAGAGTCACCATTACTTGCAAGGCAAGTGA | SEQ ID NO: 172 |
| | GGACATATATAATCGG | |
| gn33 | CTACTTGTCTGTATCTCTAGGAGGCAGAGTCACCATTACTTGCAAGGCAAGTGA | SEQ ID NO: 173 |
| | CCACATTAATAATTGG | |
| gr32 | CCAGGAAATATTCCTAAACTATTGATCTATAAGGCTTCCAACTTGCACACAGGC | SEQ ID NO: 174 |
| | GTCCCATCAAGGTTTA | |
| he24 | TATTGTGATGACGCAGGCTGCATTCTCCAATCCAGTCACTCTTGGAACATCAGC | SEQ ID NO: 175 |
| | TTCCATCTCCTGCAGG | |
| hf24 | ATATTGTGATGACTCAGGCTGCACCCTCTGTACCTGTCACTCCTGGAGAGTCAG | SEQ ID NO: 176 |
| | TATCCATCTCCTGCAG | |
| hg24 | TCTAGTAAGAGTCTCCTATATAAGGATGGGAAGACATACTTGAATTGGTTTCTG | SEQ ID NO: 177 |
| | CAGAGACCAGGACAAT | |
| if11 | TGACTTGCCAGGCAAGTCAGGGCACTAGCATTAATTTAAACTGGTTTCAGCAA | SEQ ID NO: 178 |
| | AAACCAGGGAAAGCTCC | |
| kb4 | AATTGTGCTCACTCAGTCTCCAGCCATCACAGCTGCATCTCTGGGGCAAAAGG | SEQ ID NO: 179 |
| | TCACCATCACCTGCAGT | |
| kf4 | CCCGGGGAGAAGATCACTATCACCTGCAGTGCCAGCTCAAGTATAAGTTCCAA | SEQ ID NO: 180 |
| | TTACTTGCATTGGTATC | |
| kh4 | GCTCAAGTATAAGTTCCAGCAACTTGCACTGGTACCAGCAGAAGTCAGAAACC | SEQ ID NO: 181 |
| | TCCCCCAAACCCTGGAT | |
| kj4 | TTACTTGCACTGGTACGAGCAGAAGTCAGGCGCTTCCCCCAAACCCTTGATTCA | SEQ ID NO: 182 |
| | TAGGACATCCAACCTG | |
| kk4 | CTCAAGTGTAAGTTACATGCACTGGTACCAGCAGAAGTCAGGCACCTCCCCCA | SEQ ID NO: 183 |
| | AAAGATGGATTTATGAC | |
| km4 | AGGATCCTCGCCCAAACCCTGGATTTATGACACATCCAACCTGGCTTCTGGATT | SEQ ID NO: 184 |
| | CCCTGCTCGCTTCAGT | |
| kn4 | CCAGCTCAAGTATAAGTTACATGCACTGGTACCAGCAGAAGCCAGGCACCTCC | SEQ ID NO: 185 |
| | CCCAAAAGATGGATTTA | |
| Kappa | CTTCACCCATTGTCAAGAGCTTCAACAGGAATGAGTGTTAGAGACAAAGGTCC | SEQ ID NO: 186 |
| Constant | TGAGACGCCACCACCAG | |
| Lambda1 | AGAAACATGCCCAAGTGTATCCTTGGTGCTTTTGCCTACCATAGCCCTTCTCTC | SEQ ID NO: 187 |
| Constant | TACCCTCAAAATGCAC | |
| Lambda2 | CCGTGTTTCCACCTTCCTCTGAGGAGCTCAAGGAAAACAAAGCCACACTGGTG | SEQ ID NO: 188 |
| Constant | TGTCTGATTTCCAACTT | |
| Lambda3 | AATCCCTTCTTTCATTCACACAGGTCAGCCCAAGTCCACTCCCAGACTCACCAT | SEQ ID NO: 189 |
| Constant | GTTTCCACCTTCCCCT | |
| Actin | CGTGCACCGCAAGTGCTTCTAGGCGGACTGTTACTGAGCTGCGTTTTACACCCT | SEQ ID NO: 190 |
| Beta | TTCTTTGACAAAACCT | |

TABLE 4

Additional human light chain V gene oligonucleotides. The complements of
these sequences may also be used.

| Sequence | | |
|---|---|---|
| Name | Secondary Sequence | |
| >A1_134 | AGGCCAATCTCCAAGGCGCCTAATTTATAAGGTTTCTAACTGGGACTCTGG | SEQ ID NO: 191 |
| | GGTCCCAGACAGATTCAGC | |
| >A11_93 | AGCTACTTAGCCTGGTACCAGCAGAAACCTGGCCTGGCGCCCAGGCTCCTC | SEQ ID NO: 192 |
| | ATCTATGATGCATCCAGCA | |
| >A14_28 | TCCTCTCTGTGACTCCAGGGGAGAAAGTCACCATCACCTGCCAGGCCAGTG | SEQ ID NO: 193 |
| | AAGGCATTGGCAACTACTT | |
| >A17_134 | AGGCCAATCTCCAAGGCGCCTAATTTATAAGGTTTCTAACCGGGACTCTGG | SEQ ID NO: 194 |
| | GGTCCCAGACAGATTCAGC | |
| >A18b_109 | CTATTTGTATTGGTACCTGCAGAAGCCAGGCCAGTCTCCACAGCTCCTAAT | SEQ ID NO: 195 |
| | CTATGAAGTTTCCAGCCGG | |
| >A19_159 | TATTTGGGTTCTAATCGGGCCTCCGGGGTCCCTGACAGGTTCAGTGGCAGT | SEQ ID NO: 196 |
| | GGATCAGGCACAGATTTTA | |
| >A2_109 | ATTTGTATTGGTACCTGCAGAAGCCAGGCCAGCCTCCACAGCTCCTGATCT | SEQ ID NO: 197 |
| | ATGAAGTTTCCAACCGGTT | |
| >A20_123 | AAAGTTCCTAAGCTCCTGATCTATGCTGCATCCACTTTGCAATCAGGGGTC | SEQ ID NO: 198 |
| | CCATCTCGGTTCAGTGGCA | |
| >A23_143 | TCCAAGACTCCTAATTTATAAGATTTCTAACCGGTTCTCTGGGGTCCCAGA | SEQ ID NO: 199 |
| | CAGATTCAGTGGCAGTGGG | |
| >A26_65 | CTGCCGGGCCAGTCAGAGCATTGGTAGTAGCTTACACTGGTACCAGCAGA | SEQ ID NO: 200 |
| | AACCAGATCAGTCTCCAAAG | |
| >A27_1 | AAATTGTGTTGACGCAGTCTCCAGGCACCCTGTCTTTGTCTCCAGGGGAAA | SEQ ID NO: 201 |
| | GAGCCACCCTCTCCTGCAG | |
| >A30_85 | TTAGAAATGATTTAGGCTGGTATCAGCAGAAACCAGGGAAAGCCCCTAAG | SEQ ID NO: 202 |
| | CGCCTGATCTATGCTGCATC | |
| >A5_101 | ATACACCTATTTGTATTGGTTTCTGCAGAAAGCCAGGCCAGTCTCCACACT | SEQ ID NO: 203 |
| | CCTGATCTATGAAGTTTCC | |
| >A7_222 | GATTTCACACTGAAAATCAGCAGGGTGGAAGCTGAGGATGTCGGGGTTTA | SEQ ID NO: 204 |
| | TTACTGCACGCAAGCTACAC | |
| >B2_214 | CCCTCACAATTAATAACATAGAATCTGAGGATGCTGCATATTACTTCTGTC | SEQ ID NO: 205 |
| | TACAACATGATAATTTCCC | |
| >B3_27 | TCCCTGGCTGTGTCTCTGGGCGAGAGGGCCACCATCAACTGCAAGTCCAGC | SEQ ID NO: 206 |
| | CAGAGTGTTTTATACAGCT | |
| >L1_71 | GGCGAGTCAGGGCATTAGCAATTATTTAGCCTGGTTTCAGCAGAAACCAG | SEQ ID NO: 207 |
| | GGAAAGCCCCTAAGTCCCTG | |
| >L10_92 | CAGCTACTTAACCTGGTATCAGCAGAAACCTGGCCAGGCGCCCAGGCTCCT | SEQ ID NO: 208 |
| | CATCTATGGTGCATCCACC | |
| >L11_217 | TCACCATCAGCAGCCTGCAGCCTGAAGATTTTGCAACTTATTACTGTCTAC | SEQ ID NO: 209 |
| | AAGATTACAATTACCCTCC | |
| >L12_47 | AGACAGAGTCACCATCACTTGCCGGGCCAGTCAGAGTATTAGTAGCTGGTT | SEQ ID NO: 210 |
| | GGCCTGGTATCAGCAGAAA | |
| >L14_68 | TCGGGCGAGGCAGGGCATTAGCAATTATTTAGCCTGGTTTCAGCAGAAACC | SEQ ID NO: 211 |
| | AGGGAAAGTCCCTAAGCAC | |
| >L15_92 | CTGGTTAGCCTGGTATCAGCAGAAACCAGAGAAAGCCCCTAAGTCCCTGA | SEQ ID NO: 212 |
| | TCTATGCTGCATCCAGTTTG | |
| >L16_217 | TCACCATCAGCAGCCTGCAGTCTGAAGATTTTGCAGTTTATTACTGTCAGC | SEQ ID NO: 213 |
| | AGTATAATAACTGACCTCC | |
| >L18_216 | CTCACCATCAGCAGCCTGCAGCCTGAAGATTTTGCAACTTATTACTGTCAA | SEQ ID NO: 214 |
| | CAGTTTAATAATTACCCTC | |
| >L19_206 | AGATTTCACTCTCACTATCAGCAGCCTGCAGCCTGAAGATTTTGCAACTTA | SEQ ID NO: 215 |
| | CTATTGTCAACAGGCTAAC | |
| >L2_217 | TCACCATCAGCAGCCTGCAGTCTGAAGATTTTGCAGTTTATTACTGTCAGC | SEQ ID NO: 216 |
| | AGTATAATAACTGGCCTCC | |
| >L20_193 | GTGGGCCTGGGACAGACTTCACTCTCACCATCAGCAGCCTAGAGCCTGAA | SEQ ID NO: 217 |
| | GATTTTGCAGTTTATTACTG | |
| >L22_113 | GAAACCAGGGAAATCCCCTAAGCTCTTCCTCTATGATGCAAAAGATTTGCA | SEQ ID NO: 218 |
| | CCCTGGGGTCTCATCGAGG | |
| >L23_104 | GTATCAGCAAAAACCAGCAAAAGCCCCTAAGCTCTTCATCTATTATGCATC | SEQ ID NO: 219 |
| | CAGTTTGCAAAGTGGGGTC | |
| >L24_24 | TCCTTACTCTCTGCATCTACAGGAGACAGAGTCACCATCAGTTGTCGGATG | SEQ ID NO: 220 |
| | AGTCAGGGCATTAGCAGTT | |
| >L25_92 | CAGCTACTTATCCTGGTACCAGCAGAAACCTGGGCAGGCTCCCAGGCTCCT | SEQ ID NO: 221 |
| | CATCTATGGTGCATCCACC | |
| >L4/18a_216 | CTCACCATCAGCAGCCTGCAGCCTGAAGATTTTGCAACTTATTACTGTCAA | SEQ ID NO: 222 |
| | CAGTTTAATAGTTACCCTC | |
| >L5_196 | GATCTGGGACAGATTTCACTCTCACCATCAGCAGCCTGCAGCCTGAAGATT | SEQ ID NO: 223 |
| | TTGCAACTTACTATTGTCA | |
| >L6_49 | AAAGAGCCACCCTCTCCTGCAGGGCCAGTCAGAGTGTTAGCAGCTACTTAG | SEQ ID NO: 224 |
| | CCTGGTACCAACAGAAACC | |
| >L8_215 | TCTCACAATCAGCAGCCTGCAGCCTGAAGATTTTGCAACTTATTACTGTCA | SEQ ID NO: 225 |
| | ACAGCTTAATAGTTACCCT | |
| >L9_28 | CATTCTCTGCATCTACAGGAGACAGAGTCACCATCACTTGTCGGGCGAGTC | SEQ ID NO: 226 |
| | AGGGTATTAGCAGTTATTT | |
| >O1_159 | ATCTATACGCTTTCCTATCGGGCCTCTGGAGTCCCAGACAGGTTCAGTGGC | SEQ ID NO: 227 |
| | AGTGGGTCAGGCACTGATT | |
| >O2_215 | TCTCACCATCAGCAGTCTGCAACCTGAAGATTTTGCAACTTACTACTGTCA | SEQ ID NO: 228 |
| | ACAGAGTTACAGTACCCCT | |
| >O4_217 | TCACTATCAGCAGCCTGCAGCCTGAAGATGTTGCAACTTATTACGGTCAAC | SEQ ID NO: 229 |
| | GGACTTACAATGCCCCTCC | |
| >O8_145 | ACGATGCATCCAATTTGGAAACAGGGGTCCCATCAAGGTTCAGTGGAAGT | SEQ ID NO: 230 |
| | GGATCTGGGACAGATTTTAC | |
| >V1-11_121 | CAGGAAAGGCTCCCAAACTCCTCATCTATTATGATGATCTGCTGCCCTCAG | SEQ ID NO: 231 |
| | GGGTCTCTGACCGATTCTC | |
| >V1-13_228 | ATCACTGGGCTCCAGGCTGAGGATGAGGCTGATTATTACTGCCAGTCCTAT | SEQ ID NO: 232 |
| | GACAGCAGCCTGAGTGGTT | |
| >V1-16_51 | GTCACCATCTCTTGTTCTGGAAGCAGCTCCAACATCGGAAGTAATACTGTA | SEQ ID NO: 233 |
| | AACTGGTACCAGCAGCTCC | |
| >V1-17_93 | AATTATGTATACTGGTACCAGCAGCTCCCAGGAACGGCCCCCAAACTCCTC | SEQ ID NO: 234 |
| | ATCTATAGTAATAATCAGC | |
| >V1-18_226 | CCATCACTGGACTCCAGTCTGAGGATGAGGCTGATTATTACTGCAAAGCAT | SEQ ID NO: 235 |
| | GGGATAACAGGCTGAATGC | |
| >V1-19_225 | ATCACCGGACTCCAGACTGGGGACGAGGCCGATTATTACTGCGGAACATG | SEQ ID NO: 236 |
| | GGATAGCAGCCTGAGTGCTG | |
| >V1-2_227 | CGTCTCTGGGCTCCAGGCTGAGGATGAGGCTGATTATTACTGCAGCTCATA | SEQ ID NO: 237 |
| | TGCAGGCAGCAACAATTTC | |
| >V1-20_91 | ACCAAGGAGCAGCTTGGCTGCAGCAGCACCAGGGCCACCCTGCCAAACTC | SEQ ID NO: 238 |
| | CTATCCTACAGGAATAACAA | |
| >V1-22_36 | TCTCCGGGGAAGACGGTAACCATCTCCTGCACCCGCAGCAGTGGCAGCATT | SEQ ID NO: 239 |
| | GCCAGCAACTATGTGCAGT | |
| >V1-3_227 | CATCTCTGGGCTCCAGGCTGAGGATGAGGCTGATTATTACTGCTGCTCATA | SEQ ID NO: 240 |
| | TGCAGGCAGCTACACTTTC | |
| >V1-4_143 | CATGATTTATGAGGTCAGTAATCGGCCCTCAGGGGTTTCTAATCGCTTCTCT | SEQ ID NO: 241 |
| | GGCTCCAAGTCTGGCAAC | |
| >V1-5_85 | TTGGTAGTTATAACCGTGTCTCCTGGTACCAGCAGCCCCCAGGCACAGCCC | SEQ ID NO: 242 |
| | CCAAACTCATGATTTATGA | |
| >V1-7_227 | AATCTCTGGGCTCCAGGCTGAGGACGAGGCTGATTATTACTGCTGCTCATA | SEQ ID NO: 243 |
| | TGCAGGTAGTAGCACTTTC | |
| >V1-9_83 | CGTTGGGGATTATGATCATGTCTTCTGGTACCAAAAGCGTCTCAGCACTAC | SEQ ID NO: 244 |
| | CTCCAGACTCCTGATTTAC | |
| >V2-1_30 | TCCGTGTCCCCAGGACAGACAGCCAGCATCACCTGCTCTGGAGATAAATTG | SEQ ID NO: 245 |
| | GGGGATAAATATGCTTGCT | |
| >V2-11_84 | AAAAAATATGCTTATTGGTACCAGCAGAAGCCAGGCCAGTTCCCTGTGCTG | SEQ ID NO: 246 |
| | GTGATATATAAAGACAGCG | |
| >V2-13_44 | ACAGACAGTCAGGATCACATGCCAAGGAGACAGCCTCAGAAGCTATTATG | SEQ ID NO: 247 |
| | CAAGCTGGTACCAGCAGAAG | |
| >V2-14_218 | CATCAGCAGGGTCGAAGCCGGGGATGAGGCCGACTATTACTGTCAGGTGT | SEQ ID NO: 248 |
| | GGGATAGTAGTAGTGATCAT | |
| >V2-15_125 | CCCTGAGTTGGTGATATACGAAGATAGTGAGCGGTACCCTGGAATCCCTGA | SEQ ID NO: 249 |
| | ACGATTCTCTGGGTCCACC | |
| >V2-17_204 | ACAGTCACGTTGACCATCAGTGGAGTCCAGGCAGAAGATGAGGCTGACTA | SEQ ID NO: 250 |
| | TTACTGTCAATCAGCAGACA | |
| >V2-19_211 | CCTTGACCATCAGCGGGGCCCAGGTTGAGGATGAGGCTGACTATTACTGTT | SEQ ID NO: 251 |
| | ACTCTGCGGCTGACAACAA | |
| >V2-6_215 | GACCATCAGCAGAGCCCAAGCCGGGGATGAGGCTGACTATTACTGTCAGG | SEQ ID NO: 252 |
| | TGTGGGACAGCAGCACTGCA | |
| >V2-7_220 | TCAGTGGGGCCCAGGTGGAGGATGAAGCTGACTACTACTGTTACTCAACA | SEQ ID NO: 253 |
| | GACAGCAGTGGTAATCATAG | |
| >V2-8_189 | TCCAACCCAGGGAACACCGCCACCCTAACCATCAGCAGGATCGAGGCTGG | SEQ ID NO: 254 |
| | GGATGAGGCTGACTATTACT | |
| >V3-2_224 | GACACTGTCAGGTGTGCAGCCTGAGGACGAGGCTGAGTATTACTGCCTGCT | SEQ ID NO: 255 |
| | CTACTATGGTGGTGCTCAG | |
| >V3-3_95 | TCATTATCCCTACTGGTTCCAGCAGAAGCCTGGCCAAGCCCCCAGGACACT | SEQ ID NO: 256 |
| | GATTTATGATACAAGCAAC | |
| >V3-4_209 | GAACAAAGCTGCCCTCACCATCACGGGGGCCCAGGCAGATGATGAATCTG | SEQ ID NO: 257 |
| | ATTATTACTGTGTGCTGTAT | |
| >V4-1_20 | ACCTTCCTCCTCCGCATCTCCTGGAGAATCCGCCAGACTCACCTGCACCTT | SEQ ID NO: 258 |
| | GCCCAGTGACATCAATGTT | |
| >V4-2_110 | GTACCAGCAGAAGCCAGGGAGTCCTCCCCAGTATCTCCTGAGGTACAAAT | SEQ ID NO: 259 |
| | CAGACTCAGATAAGCAGCAG | |
| >V4-3_110 | GTACCAGCAGAAGCCAGAGAGCCCTCCCCGGTATCTCCTGAGCTACTACTC | SEQ ID NO: 260 |
| | AGACTCAAGTAAGCATCAG | |
| >V4-4_66 | ATGCTGAGCAGTGGCTTCAGTGTTGGGGACTTCTGGATAAGGTGGTACCAA | SEQ ID NO: 261 |
| | CAAAAGCCAGGGAACCCTC | |
| >V4-6_182 | ACCTGGGGTCCCCAGTCGAGTCTCTGGCTCCAAGGAGACCTCAAGTAACAC | SEQ ID NO: 262 |
| | AGCGTTTTTGCTCATCTCT | |
| >V5-1_77 | TGAGCACAGCACCTACACCATCGAATGGTATCAACAGAGACCAGGGAGGT | SEQ ID NO: 263 |
| | CCCCCCAGTATATAATGAAG | |
| >V5-2_237 | ATCAAGAACATCCAGGAAGAAGATGAGAGTGACTACCACTGTGGGGCAGA | SEQ ID NO: 264 |
| | CCATGGCAGTGGGAGCAACT | |
| >V5-4_117 | CCAGGGAAGGCCCCTCGGTACTTGATGAAGCTTGAAGGTAGTGGAAGCTA | SEQ ID NO: 265 |
| | CAACAAGGGGAGGGGAGTTC | |
| >V5-6_120 | GAGAAGGGCCCTCGGTACTTGATGAAGCTTAACAGTGATGGCAGCCACAG | SEQ ID NO: 266 |
| | CAAGGGGGACGGGATCCCTG | |

TABLE 5

Additional mouse light chain V gene selected oligonucleotides. The
complements of these sequences may also be used.

Secondary Sequence

| | | |
|---|---|---|
| 12-38-65 | CATGTCGAGCAAGTGAGAACATTTACTACAGTTTAGCATGGTATCAGCAGAAGC | SEQ ID NO: 267 |
| | AAGGGAAATCTCC | |
| 12-41-86 | TTCACAATTATTTAGCATGGTATCAGCAGAAACAGGGAAAATCTCCTCAGGTCCT | SEQ ID NO: 268 |
| | GGTCTATAATGC | |
| 12-44-70 | CGAGCAAGTGAGAATATTTACAGTTATTTAGCATGGTATCAGCAGAAACAGGGA | SEQ ID NO: 269 |
| | AAATCTCCTCAGC | |
| 12-46-70 | CGAGCAAGTGAGAATATTTACAGTAATTTAGCATGGTATCAGCAGAAACAGGGA | SEQ ID NO: 270 |
| | AAATCTCCTCAGC | |
| 19-13-103 | TGGTATCAACAGAAACCAGGACAATCTCCTAAACTACTGATTTACTCGGCATCC | SEQ ID NO: 271 |
| | AATCGGTACACTG | |
| 19-14-197 | GATCTGGGACAGATTTCACTCTCACCATTAGCAATGTGCAATCTGAAGACCTGG | SEQ ID NO: 272 |
| | CAGATTATTTCTG | |
| 19-15-217 | CTCACCATCAGCAATGTGCAGTCTGAAGACTTGGCAGAGTATTTCTGTCAGCAAT | SEQ ID NO: 273 |
| | ATAACAGCTATC | |
| 19-17-215 | CTTTCACCATCAGCAGTGTGCAGGCTGAAGACCTGGCAGTTTATTACTGTCAGCA | SEQ ID NO: 274 |
| | ACATTATAGTAC | |
| 19-20-69 | CAAGGCCAGTGAGAATGTGGGTACTTATGTATCCTGGTATCAACAGAAACCAGA | SEQ ID NO: 275 |
| | GCAGTCTCCTAAA | |
| 19-23-206 | CAGATTTCACTCTCACCATTAGCAATGTGCAGTCTGAAGACTTGGCAGATTATTT | SEQ ID NO: 276 |
| | CTGTCAGCAATATAG | |
| 19-25-209 | ATTATACTCTCACCATCAGCAGTGTGCAGGCTGAAGACCTGGCACTTTATTACTG | SEQ ID NO: 277 |
| | TCAGCAACATTATA | |
| 19-29-69 | CAAGGCCAGTGAGAATGTGGGTACTTATGTATCCTGGTATCAACAGAAACCAGA | SEQ ID NO: 278 |
| | GCAGTCTCCTAAA | |
| 19-32-124 | CAGTCTCCTAAACTGCTGATATACTATGCATCCAATCGCTACACTGGAGTCCCTG | SEQ ID NO: 279 |
| | ATCGCTTCACTG | |
| 21-1-117 | GTACCAACAGAAACCAGGACAGCCACCCAAACTCCTCATCTATGCTGCATCCAA | SEQ ID NO: 280 |
| | CGTAGAATCTGGG | |
| 21-10-210 | GTCTAGGACAGACTTCACCCTCACCATTGATCCTGTGGAGGCTGATGATGCTGCA | SEQ ID NO: 281 |
| | ACCTATTACTGT | |
| 21-12-117 | GTACCAACAGAAACCAGGACAGCCACCCAAACTCCTCATCTATCTTGCATCCAA | SEQ ID NO: 282 |
| | CCTAGAATCTGGG | |
| 21-2-229 | CTCAACATCCATCCTATGGAGGAGGATGATACTGCAATGTATTTCTGTCAGCAA | SEQ ID NO: 283 |
| | AGTAAGGAGGTTC | |
| 21-3-66 | CTGCAGAGCCAGCCAGAGTGTCGATTATAATGGAATTAGTTATATGCACTGGTT | SEQ ID NO: 284 |
| | CCAACAGAAACCA | |
| 21-4-117 | GTACCAACAGAAACCAGGACAGCCACCCAAACTCCTCATCTATGCTGCATCCAA | SEQ ID NO: 285 |
| | TCTAGAATCTGGG | |
| 21-5-196 | TTCAGTGGCAGTGGGTCTAGGACAGACTTCACCCTCACCATTAATCCTGTGGAG | SEQ ID NO: 286 |
| | GCTGATGATGTTG | |
| 21-7-229 | CTCAACATCCATCCTGTGGAGGAGGAGGATACTGCAACATATTACTGTCAGCAC | SEQ ID NO: 287 |
| | AGTTGGGAGATTC | |
| 21-9-212 | CTGAGTCAGACTTCACTCTCACCATCGATCCTGTGGAGGAAGATGATGCTGCAA | SEQ ID NO: 288 |
| | TGTATTACTGTAT | |
| 22-33-217 | TCTGGGACAGATTTCACTCTGACCATCAGCAGTGTACAGGTTGAAGACCTCACA | SEQ ID NO: 289 |
| | CATTATTACTGTG | |
| 23-37-81 | GAGTATTTACAAGAACCTACACTGGTATCAACAGAAATCACATCGGTCTCCAAG | SEQ ID NO: 290 |
| | GCTTCTCATCAAGTATG | |
| 23-39-202 | GGGTCAGATTTCACTCTCAGTATCAACAGTGTGGAACCTGAAGATGTTGGAGTG | SEQ ID NO: 291 |
| | TATTACTGTCAAA | |
| 23-43-195 | TGGATCAGGGACAGATTTCACTCTCAGTATCAACAGTGTGGAGACTGAAGATTT | SEQ ID NO: 292 |
| | TGGAATGTATTTCT | |
| 23-45-76 | AGTCAAAGTATTAGCAACTACCTACACTGGTATCAACAAAAATCACATGAGTCT | SEQ ID NO: 293 |
| | CCAAGGCTTCTCA | |
| 23-48-184 | TTTAGTGGCAGTGGATCAGGGACAGATTTTACTCTTAGCATCAACAGTGTGGAG | SEQ ID NO: 294 |
| | TCTGAAGATATTG | |
| 4-50-103 | TACCAGCAGAAGTCAGATGCCTCCCCCAAACTATGGATTTATTACACATCCAAC | SEQ ID NO: 295 |
| | CTGGCTCCTGGAG | |
| 4-51-207 | GATCTCTTACTCTCTCACAATCAGCAGCATGGAGGCTGAAAATGATGCAACTTAT | SEQ ID NO: 296 |
| | TACTGCCAGCAG | |
| 4-57-210 | CTCTTACTCTCTCACAATCAGCAGTGTGGAGGCTGAAGATGCTGCCACTTATTAC | SEQ ID NO: 297 |
| | TGCCAGCAGTAC | |
| 8-16-170 | CATCTGATAGGTACTCTGGAGTCCCTGATCGTTTCATAGGCAGTGGATCTGTGAC | SEQ ID NO: 298 |
| | AGATTTCACTCT | |
| 8-19 | GAGAGAAGGTCACTATGAGCTGCAAGTCCAGTCAGAGTCTGTTAAACAGTGGAA | SEQ ID NO: 299 |
| | ATCAAAAGAACTA | |
| 8-21 | GAAGGTCACTATGAGCTGCAAATCCAGTCAGAGTCTGCTCAACAGTAGAACCCG | SEQ ID NO: 300 |
| | AAAGAACTACTTG | |
| 8-24 | GACAGAAGGTCACTATGAGCTGCAAGTCCAGTCAGAGCCTTTTAAATAGTAGCA | SEQ ID NO: 301 |
| | ATCAAAAGAACTA | |
| 8-27 | GCAGGAGAAAAGGTCACTATGAGCTGTAAGTCCAGTCAAAGTGTTTTATACAGT | SEQ ID NO: 302 |
| | TCAAATCAGAAGAAC | |
| 8-28 | GAGAGAAGGTCACTATGAGCTGCAAGTCCAGTCAGAGTCTGTTAAACAGTGGAA | SEQ ID NO: 303 |
| | ATCAAAAGAACTA | |
| 8-30 | TTGGAGAGAAGGTTACTATGAGCTGCAAGTCCAGTCAGAGCCTTTTATATAGTA | SEQ ID NO: 304 |
| | GCAATCAAAAGAACTACTT | |
| 8-34-143 | GATCTCCTAAAATGCTGATAATTTGGGCATCCACTAGGGTATCTGGAGTCCCTGA | SEQ ID NO: 305 |
| | TCGCTTCATAGG | |
| RF-40 | TCTCCTGGAGAAACCATTACTATTAATTGCAGGGCAAGTAAGAGCATTAGCAAA | SEQ ID NO: 306 |
| | TATTTAGCCTGGTATCAAG | |
| aa4-1 | CAAATTGTTCTCACCCAGTCTCCAGCAATCATGTCTGCATCTCCAGGGGAGAAG | SEQ ID NO: 307 |
| | GTCACCATATCCT | |
| ac4-197 | CTGGAAACTCTTACTCTCTCACGATCAGCAGCATGGAGGCTGAAGATGTTGCCA | SEQ ID NO: 308 |
| | CTTATTACTGTTT | |
| ad4-26 | CAATCATGTCTGCATCTCCTGGGGAGAAGGTCACCATGACCTGCAGTGCCAGAT | SEQ ID NO: 309 |
| | CAAGTGTAAGTTC | |
| ae4-1 | CAAATTGTTCTCACCCAGTCTCCAGCAATCATGTCTGCATCTCCTGGGGAGAAGG | SEQ ID NO: 310 |
| | TCACCTTGACCT | |
| af4-120 | CACTTCTCCCAAACTCTTGATTTATAGCACATCCAACCTGGCTTCTGGAGTCCCT | SEQ ID NO: 311 |
| | TCTCGCTTCAGT | |
| ag4-128 | CTTCTACCAAATTCTGGATTTATAGGACATCCAACCTGGCTTCAGAAGTCCCAGC | SEQ ID NO: 312 |
| | TCCCTTCAGTGG | |
| ah4-125 | GATCCTCCCCAAAACTCTGGATTTATAGCATATCCAACCTGGCTTCTGGAGTCCC | SEQ ID NO: 313 |
| | AGCTCGCTTCAG | |
| ai4-1 | CAAATTGTTCTCACCCAGTCTCCAGCAATCATGTCTGCATCTCTAGGGGAACGGG | SEQ ID NO: 314 |
| | TCACCATGACCT | |
| aj4-119 | GATCCTCCCCCAAACTCTGGATTTATAGCATATCCAACCTGGCTTCTGGAGTCCC | SEQ ID NO: 315 |
| | TGCTCGCTTCAG | |
| al4-133 | CTCTGGATTTATTTAACATTCAACTTGGCTTCTGGAGTCCCTGCTCGCTTCAGTGG | SEQ ID NO: 316 |
| | CAGTGGGTCTG | |
| am4-212 | CTCTCACAATCAGCAGAGTGGAGGCTGAAGATGCTGCCACTTATTACTGCCAGC | SEQ ID NO: 317 |
| | AGTGGAGTAGTAA | |
| an4-203 | CATCTTTCTCTTTCACAATCAACAGCATGGAGGCTGAAGATGTTGCCACTTATTA | SEQ ID NO: 318 |
| | CTGTCAGCAAAG | |
| ap4-80 | CAAGTGTAAGTTACATGCACTGGTTCCAGCAGAAGCCAGGCACTTCTCCCAAAC | SEQ ID NO: 319 |
| | TCTGGATTTATAG | |
| aq4-38 | CATCTCGAGGGGAGAAGGTCACCATGACCTGCAGTGCCAGCTCAAGTGTAAGTT | SEQ ID NO: 320 |
| | ACATGTACTGGTA | |
| ar4-180 | CTTCAGTGGCAGTGGATCTGGGACCTCTTATTCTCTCACAATCAGCAGCATGGAG | SEQ ID NO: 321 |
| | GCTGAAGATGCT | |
| at4-38 | CATCTCCAGGGGAGAAGGTCACCATGACCTGCAGTGCCAGCTCAAGTGTAAGTT | SEQ ID NO: 322 |
| | ACATGTACTGGTA | |
| ay4-44 | GAGGGGAGAAGGTCACCATCACCTGCCGTGCCAGCTCAAGTATAAGTTCCAATT | SEQ ID NO: 323 |
| | ACTTACACTGGTA | |
| ba9-195 | TGGATCTGGGCAAGATTATTCTCTCACCATCAGCAGCCTGGAGTATGAAGATAT | SEQ ID NO: 324 |
| | GGGAATTTATTATT | |
| bb1-60 | CATCTCTTGCAGATCTAGTCAGAGCCTTGTACACAGTAATGGAAACACCTATTTA | SEQ ID NO: 325 |
| | CATTGGTACCTG | |
| bb1.1-59 | CCATCTCTTGCAGATCTAGTCAGAGCCTTGTACACAGTAATGGAAACACCTATTT | SEQ ID NO: 326 |
| | ATATTGGTACCTG | |
| bd2-85 | CTCTTAGATAGTGATGGAAAGACATATTTGAATTGGTTGTTACAGAGGCCAGGC | SEQ ID NO: 327 |
| | CAGTCTCCAAAGC | |
| bi2-172 | AAACTGGACCCTGGCATCCCTGACAGGTTCAGTGGCAGTGGATCAGAAACAGAT | SEQ ID NO: 328 |
| | TTTACACTTAAAAT | |
| bj2-108 | CTATTTGAATTGGTTATTACAGAGGCCAGGCCAGTCTCCAAAGCGCCTAATCTAT | SEQ ID NO: 329 |
| | CTGGTGTCTAAA | |
| bl1-57 | CTCCATCTCTTGCAGGTCTAGTCAGAGCCTTGAAAACAGTAATGGAAACACCTA | SEQ ID NO: 330 |
| | TTTGAACTGGTAC | |
| bt20-123 | GGAACCTCCTAAGCTCCTTATTTCAGAAGGCAATACTCTTCGTCCTGGAGTCCCA | SEQ ID NO: 331 |
| | TCCCGATTCTCC | |
| bv9-81 | GGACATTGGTAGTAGCTTAAACTGGCTTCAGCAGGAACCAGATGGAACTATTAA | SEQ ID NO: 332 |
| | ACGCCTGATCTAC | |
| bw20-123 | GGAACCTCCTAAGCTCCTTATTTCAGAAGGCAATACTCTTCGTCCTGGAGTCCCA | SEQ ID NO: 333 |
| | TCCCGATTCTCC | |
| cb9-42 | TCTGGGAGACAGAATAACCATCACTTGCCAGGCAACTCAAGACATTGTTAAGAA | SEQ ID NO: 334 |
| | TTTAAACTGGTAT | |
| ce9-184 | TTCAGTGGCAGTGGGTCTGGAACAGATTATTCTCTCACCATTAGCAACCTGGAGC | SEQ ID NO: 335 |
| | AAGAAGATATTG | |
| cf9-109 | CAGCAGAAACCAGGGAAATCATTTAAGGGCCTGATCTATCATGGAACCAACTTG | SEQ ID NO: 336 |
| | GAAGATGGAGTTC | |
| ci12-81 | GACCATTGGTACATGGTTAGCATGGTATCAGCAGAAACCAGGGAAATCTCCTCA | SEQ ID NO: 337 |
| | GCTCCTGATTTAT | |
| cj9 | ACCTGATCTATGAAACATCCAATTTAGATTCTGGTGTCCCAAAAAGGTTCAGTGG | SEQ ID NO: 338 |
| | CAGTAGGTCTGG | |
| cp9 | TTATTCTCTCACCATCAGCAACCTGGAACCTGAAGATATTGCCACTTACTATTGT | SEQ ID NO: 339 |
| | CAGCAGTATAGT | |
| cr1 | GGAGATCAAGCCTCCATCTCTTGCAGATCTAGTCAGAGCATTGTACATAGTAAT | SEQ ID NO: 340 |
| | GGAAACACCTATTTAGAAT | |
| cs1 | GATTTCACACTCAAGATCAGCACAATAAAGCCTGAGGACTTGGGAATGTATTAC | SEQ ID NO: 341 |
| | TGCTTACAAGGTA | |
| cv1 | CTCAAGATCAGCAGAGTGGAGGCTGAGGATTTGGGAGTTTATTATTGCTTCCAG | SEQ ID NO: 342 |
| | AGTAACTATCTTC | |
| cw9 | GGAAATTAGTGGTTACTTAAGCTGGCTTCAGCAGAAACCAGATGGAACTATTAA | SEQ ID NO: 343 |
| | ACGCCTGATCTAC | |
| cy9 | CTAGTCAGGGCATTAGAGGTAATTTAGACTGGTATCAGCAGAAACCAGGTGGAA | SEQ ID NO: 344 |
| | CTATTAAACTCCTG | |
| dv-36 | GAAACAACACAGGCTCCAGCTTCTCTGAGTTTTTCTCTTGGTGAAACAGCAACAC | SEQ ID NO: 345 |
| | TGTCATGCAGGTC | |
| fl12 | CAAGTGAGAATATTTACGGTGCTTTAAATTGGTATCAGCGGAAACAGGGAAAAT | SEQ ID NO: 346 |
| | CTCCTCAGCTCCT | |
| gj38c | CTGCTCATACATTACACATCTACATTACAGCCAGGCATCCCATCAAGGTTCAGTG | SEQ ID NO: 347 |
| | GAAGTGGGTCTG | |
| gm33 | GTTCCTTCAAGATTCAGTGGCAGTGGATCTGGAAAGGATTACACTCTCAGCATTA | SEQ ID NO: 348 |
| | CCAGTCTTCAGA | |
| gn33 | GACATCCAGATGACACAATCTTCATCCTACTTGTCTGTATCTCTAGGAGGCAGAG | SEQ ID NO: 349 |
| | TCACCATTACTT | |
| gr32 | GGAAATATTCCTAAACTATTGATCTATAAGGCTTCCAACTTGCACACAGGCGTCC | SEQ ID NO: 350 |
| | CATCAAGGTTTA | |
| he24 | GATATTGTGATGACGCAGGCTGCATTCTCCAATCCAGTCACTCTTGGAACATCAG | SEQ ID NO: 351 |
| | CTTCCATCTCCT | |
| hf24 | CTGAGAATCAGTAGAGTGGAGGCTGAGGATGTGGGTGTTTATTACTGTATGCAA | SEQ ID NO: 352 |
| | CATCTAGAATATCC | |
| hg24 | CTGGAAATCAGTAGAGTGAAGGCTGAGGATGTGGGTGTGTATTACTGTGAACAA | SEQ ID NO: 353 |
| | CTTGTAGAGTATC | |
| if11 | TCTCACCATCAGCAGCCTGGAGGATGAAGATATGGCAACTTATTTCTGTCTACAG | SEQ ID NO: 354 |
| | CATAGTTATCTC | |
| kb4 | CATGGATTTATGAAATATCCAAACTGGCTTCTGGAGTCCCAGCTCGCTTCAGTGG | SEQ ID NO: 355 |
| | CAGTGGGTCTGG | |
| kf4 | CAATTACTTGCATTGGTATCAGCAGAAGCCAGGATTCTCCCCTAAACTCTTGATT | SEQ ID NO: 356 |
| | TATAGGACATCC | |
| kh4 | CTTCTGGAGTCCCTGTTCGCTTCAGTGGCAGTGGATCTGGGACCTCTTATTCTCTC | SEQ ID NO: 357 |
| | ACAATCAGCAG | |
| kj4 | GGTCTGGGACCTCTTACTCTCTCACAATCAGCAGCGTGGAGGCTGAAGATGATG | SEQ ID NO: 358 |
| | CAACTTATTACTG | |
| kk4 | CCCAAAAGATGGATTTATGACACATCCAAACTGGCTTCTGGAGTCCCTGCTCGCT | SEQ ID NO: 359 |
| | TCAGTGGCAGTG | |
| km4 | CAAATTCTTCTCACCCAGTCTCCAGCAATCATGTCTGCATCTCCAGGGGAGAAG | SEQ ID NO: 360 |
| | GTCACCATGACCT | |
| kn4 | CCCAAAAGATGGATTTATGACACATCCAAACTGGCTTCTGGAGTCCCTGCTCGCT | SEQ ID NO: 361 |
| | TCAGTGGCAGTG | |
| ko4 | CTTCAGTGGCAGTGGATCTGGGACCTCTTATTCTCTCACAATCAGCAGCATGGAG | SEQ ID NO: 362 |
| | GCTGAAGATGCT | |
| VL1 | AAGTACTGGGGCTGTTACAACTAGTAACTATGCCAACTGGGTCCAAGAAAAACC | SEQ ID NO: 363 |
| | AGATCATTTATTC | |
| VL2 | GTACTGGGGCTGTTACAACTAGTAACTATGCCAACTGGGTTCAAGAAAAACCAG | SEQ ID NO: 364 |
| | ATCATTTATTCACT | |
| VLx | TTAGCATTTCCAACATCCAGCCTGAAGATGAAGCAATATACATCTGTGGTGTGG | SEQ ID NO: 365 |
| | GTGATACAATTAA | |

One of skill in the art appreciates that whether an oligonucleotide is “capable of hybridizing” to another polynucleotide depends in part on the stringency of the conditions used during hybridization. As used herein “capable of hybridizing” to a polynucleotide encoding the light chain variable region, or the complement thereof, is one that hybridizes under high stringency conditions. In the Examples, high stringency hybridization was carried out at 45° C. in a buffer containing 50% formamide, 5×SSC, 0.1% SDS and 0.1 mg/mL BSA. After hybridization, the microarrays were washed in 2×SSC, 0.1% SDS at 42° C. for 5 minutes, two times in 1×SSC at room temperature, two times in 0.1×SSC, and in water for 30 seconds. One of skill in the art would appreciate that the hybridization and washing conditions can be altered while maintaining high stringency conditions.

Oligonucleotides corresponding to the sequences in Table 2 and Table 3 were generated and printed onto a glass slide to form the microarray used in the Examples. One of skill in the art would appreciate that a microarray having a subset of the oligonucleotides of Tables 2 and 3 may also be useful. For example, a microarray comprising a subset of oligonucleotides capable of hybridizing to a polynucleotide comprising a sequence encoding at least a portion of a light chain variable region that is associated with a disease, or a complement thereof, may be used in the methods of the invention. The subset of oligonucleotides capable of hybridizing to the light chain variable regions associated with a systemic autoimmune disease, such as the light chain variable regions listed in Table 1, may also be useful in the methods of the invention. One of skill in the art would also appreciate that two or more oligonucleotides capable of hybridizing to a single light chain variable gene could be used in the microarray. Use of multiple oligonucleotides specific for the same gene improves resolution and minimizes problems with cross-hybridization.

In addition to the oligonucleotides capable of hybridizing to the light chain variable regions, or complements thereof, appropriate quality control reporter oligonucleotides may be included in the microarrays of the present invention. Tables 2 and 3 include several oligonucleotides that were used as controls in the Examples. These include oligonucleotides capable of hybridizing to polynucleotides encoding beta actin, CD19, CD20, the kappa constant region and several lambda constant regions. The controls chosen for use in the Examples are not limiting. One of skill in the art could design control oligonucleotides from a wide variety of cellular genes.

Each oligonucleotide species used is immobilized at a distinct location or domain on a substantially planar solid surface of a substrate to form a microarray. Any suitable substrate may be used, including, but not limited to, glass, silicon, nitrocellulose, paper or other solid surface materials. The oligonucleotide species can be RNA or DNA. The oligonucleotide species can be immobilized by depositing or synthesizing oligonucleotides at specific locations on the microarray by methods known to those of skill in the art. Generally each oligonucleotide species is present in replicates on the microarray. Alternatively, pools of multiple oligonucleotide species could be used. In the Examples, each oligonucleotide species was printed either six times or ten times in distinct locations to serve as an internal control for even hybridization of the target polynucleotides to the slide. The replicate oligonucleotide species can be printed near each other, in a set pattern or randomly on the microarray. This generates a microarray chip that serves as a platform for identification and quantification of light chain variable region usage.

In the Examples, the microarray was used to detect the expression of light chain variable genes in B cells. However, the microarrays could also be used to detect light chain variable gene expression in plasma cells or plasmablasts. The cells may be harvested from any source, as long as the cell sample contains B cells. Peripheral blood is one source for obtaining cells from the subject. Cells may also be harvested from a body fluid of the subject, including, but not limited to synovial fluid, cerebrospinal fluid, lymph, bronchioalveolar lavage fluid, gastrointestinal secretions, saliva, urine, and tears. The cells may also be derived from a tissue of the individual, e.g., by performing a tissue biopsy on tissues, including, but not limited to, the spleen and lymph nodes. When assaying for a particular disease condition the selection of appropriate cell sources will be apparent to those of ordinary skill in the art. For example, to assay for autoimmune disorders affecting the joints (e.g., rheumatoid arthritis), synovial fluid is a suitable source of cells. In a patient with multiple sclerosis, cerebral spinal fluid is a suitable source of cells. In the Examples, the B cells were harvested from cerebral spinal fluid and peripheral blood.

Fluorescent activated cell sorting (FACS) was used in the Examples to harvest and select B cells by expression of specific cell surface markers, namely CD19 and CD20, and lack of expression of other markers that are indicative of plasma cells, memory B cells and plasmablasts, namely CD138, CD27 and CD38. One of skill in the art will appreciate that other methods of sorting cells may be used, including, but not limited to, magnetic cell sorting, and density gradient centrifugation.

In the Examples, about 100 of the relevant B cells were pooled as a sample. One of skill in the art appreciates that the number of B cells used can be as few as one or as many as millions. Use of about 100 B cells produced a representative sample of the B cell light chain variable repertoire with little risk of contamination by plasma cells and required only a minimal level of amplification for detection in the microarray.

Contamination of the B cell samples by plasma cells is a concern because the concentration of light chain mRNA in plasma cells is several thousand fold higher than that of B cells. Contamination by a single plasma cell significantly biases the results of the microarray experiment. The FACS protocol used in the Examples was developed to minimize the chance of plasma cell contamination, but any suitable method of separating plasma cells from the B cells could be used. To reduce plasma cell contamination, after the B cells were sorted and RNA extracted, each sample was tested for the presence of plasma cells using RT-PCR to rule out plasma cell contamination. Importantly, this RT-PCR procedure was optimized using a single cell RT-PCR approach to detect even a single plasma cell in a sample of 100 cells. Samples with detectable plasma cell contamination were not used.

RNA may be harvested from the B cells by any suitable method. In the examples, sufficient amounts of nucleic acid for downstream applications was generated from only 100 cells by amplifying the target nucleic acid using an established antisense RNA (aRNA) amplification protocol. Alternatively, cDNA or amplified cDNA could be generated and amplified using any suitable method.

The resulting target polynucleotides were then labeled with a marker. In the Examples, a fluorescent marker was added to the target polynucleotides. Amplified target polynucleotides can be labeled by any suitable method. For example, labeled nucleotides such as biotinylated UTP or CTP can be incorporated during in vitro transcription. Labeling target molecules may occur after the amplification reaction e.g., by enzymatically modifying the 5′ end of the amplified nucleic acids. The label may be any label known to those of skill in the art, suitably the label is a fluorescent label, a radioactive label, or a luminescent label.

The labeled target polynucleotides are then contacted with the microarray under suitable hybridization conditions. Hybridization buffers and conditions may be altered to increase or decrease the stringency of the conditions as is well-known to those of skill in the art. After hybridization and washing, the microarray was analyzed for presence of bound target polynucleotide by assessing the presence of the label using a commercially available microarray scanner, such as the Axon GenePix 4000B produced by Molecular Devices or another comparable microarray scanner. Commercially available computer programs may be used to analyze the data.

Several methods are also provided for using the microarray described herein. The microarray may be used to identify light chain variable genes associated with a particular disease by comparing the light chain variable gene usage in subjects with a particular disease to subjects that do not have the disease. Such an analysis may allow identification of light chain variable genes whose expression correlates with the disease in subjects. Diseases that may correlate to particular light chain variable gene usage include, but are not limited to, systemic autoimmune diseases, cancer, especially B cell cancers, such as multiple myeloma, and immunodeficiency diseases. Systemic autoimmune diseases include, but are not limited to, systemic lupus erythematosus, multiple sclerosis, rheumatoid arthritis, scleroderma, Sjogren's syndrome, amylodosis, psoriasis, mixed connective tissue disease, polymyositis, dermatomyositis, thrombocytopenia, Wegener's granulomatosis, and autoimmune nephritis.

After expression of a particular light chain variable gene is identified as correlating with a disease, the expression of the light chain variable genes may be used to diagnose the disease, monitor disease progression, aid in prognosis, identify likely or potential sequelae of the disease associated with a particular light chain variable gene, predict the etiology of the disease or the response of the disease to particular forms of therapy. For example, a disease could be diagnosed if the pattern of detected hybridization complexes of the subject tested resembles the pattern of detected hybridization complexes of a diseased subject. As mentioned above, light chain variable gene B2 is associated with formation of amyloid fibrils in 10% of multiple myeloma patients. As an example, the microarray could be used to determine if individuals suffering from multiple myeloma are expressing light chain variable gene B2 using the microarray and tailor treatment options and determine disease prognosis based on the results.

As one of skill in the art will appreciate, expression of a particular light chain variable gene may be evaluated by any suitable means. For example, expression could be measured directly by measuring hybridization to an oligonucleotide encoding the light chain variable gene, or a complement thereof. Either the oligonucleotide or the target sample may be detectably labeled to visualize hybridization, and hybridization may be performed in any suitable format. Alternatively, expression may be detected by performing real time PCR on the target DNA using a pair of primers that hybridize to sequences within, partially overlapping or flanking the sequence encoding the light chain variable gene. Once a particular light chain variable gene of interest is identified, primer pairs may be designed using available sequence information.

The present invention also provides methods of evaluating the ability of a therapeutic agent to alter the expression of a light chain variable gene or the repertoire as a whole. First, the light chain variable gene expression of a subject with a disease is assessed using the microarray. Then the subject is treated with the therapeutic agent or undergoes a therapeutic treatment. The light chain variable gene expression is assessed again after treatment and compared to the light chain variable expression prior to treatment to determine whether the therapeutic agent or treatment affected the light chain variable repertoire. A change in light chain variable expression is indicative of effectiveness of the therapeutic agent or treatment.

The present invention also provides kits for performing the methods described herein. A kit may comprise a microarray comprising oligonucleotide species capable of hybridizing to a sequence encoding at least a portion of a light chain variable region, or a complement thereof. Suitably kits may also comprise antibodies used to sort for B cells, primers for generating the target polynucleotides, reagents needed to label the target polynucleotides and/or other reagents necessary to perform the methods described herein.

The following examples are meant to be illustrative only and are not intended as a limitation on the concepts and principles of the invention.

### EXAMPLES

Oligonucleotide sequence selection. There are 82 human and 99 mouse functional light chain variable genes. In humans, 6 pairs have identical sequences, i.e., they are duplicate genes, and are not distinguishable. There are reports of pseudogenes in both mouse and human, but these genes were not included because they are considered to be nonfunctional. However, these and other genes may be included if they are found to be misclassified and are indeed functional. Oligonucleotides specific for each of the functional mouse and human light chain variable genes were selected from the genetic sequences that are available on the NCBI website under the heading “Ig Germline Genes”.

Unique sequences ranging from 65-70 base pairs from each V region light chain (both kappa and lambda) were identified by genome scans of germline sequences. The sequence length was chosen to allow for use of high stringency hybridization conditions and thus optimize the specificity. The oligonucleotide set used in the microarray experiments described herein is shown in Table 2 and Table 3. The oligonucleotides were chosen to have minimal cross-hybridization with other variable light chain genes, to have melting temperatures of 70° C.+/−3° C. and a G-C content of 35% to 55%. The oligonucleotides were also selected to have low potential to self-fold, therefore maximizing their target size for spotting onto the slide. See Wang et al. Genome Biology 4:R5 (2003), which is incorporated herein by reference in its entirety. The following computer programs were also used in selection of the oligonucleotides:

1. Oligowiz

2. Array designer

3. NCBI mouse gene database

4. Blast

5. Mfold

6. Repeatmasker

7. Bioperl Project

8. EMBOSS.

In addition to the light chain variable region oligonucleotides, positive and negative control oligonucleotides were selected based on the same criteria. The kappa and lambda constant region oligonucleotides were used to normalize the samples for the amount of light chain present in each sample. Other control oligonucleotides included Beta actin, CD19, CD20, B220, CD 138, and Blimp-1.

Preparation of the microarray. Each of the oligonucleotides listed in Table 2 and Table 3 was generated (Integrated DNA Technologies, Coralville, Iowa). These oligonucleotides were suspended in microarray printing buffer (150 mM sodium phosphate) and printed at the University of Illinois, Urbana-Champagne using an OmniGrid 100 Microarrayer (Gene Machines, San Carlos, Calif.) onto an UltraGAPS Coated Slide (Corning, Acton, Mass.). Both positive control (CD19, CD20, B220, actin and GAPDH) and negative control (CD138, blank and Blimp-1) genes were incorporated into the microarray. Each oligonucleotide was printed in ten replicates onto a glass slide (either randomly or next to each other) and stored in vacuum sealed packaging until ready for use. Before the sample was applied to the microarray, the microarray was prehybridized in 5×SSC, 0.1% SDS and 0.1 mg/mL BSA at 42° C. for 45 minutes.

Isolation of B cells. B cells were sorted, based on the cell phenotype of CD19+ CD20+CD138− (mouse B cells sorts used CD19+CD138−), using fluorescent activated cell sorting (FACS). Human B cells were sorted by gating on CD19+, CD20+, CD138− cells. Mouse B cells were sorted by gating on CD19+, CD 138− cells. Cells were sorted directly into RNAlater (Ambion, Austin, Tex.) which prevents RNA degradation and allows samples to be stored indefinitely.

Plasma cells express CD138 and are a source of potential contamination because they express 1,000-10,000 fold more light chain than B cells and a single plasma cell could mask differential light chain variable region expression. Thus, several additional measures were taken to ensure that plasma cells were not present in the samples. First, the FACS selects against incorporation of plasma cells by selecting only CD138− cells. Additionally, only 100 cells are sorted into one sample (but many samples are collected from one individual) to minimize contamination. Finally, a reverse transcriptase-polymerase chain reaction (RT-PCR) capable of detecting plasma cell specific gene expression with single cell sensitivity was utilized to ensure the samples were plasma cell free. The PCR detects plasma cell-specific Blimp-1 gene expression (forward primer: TCTGTTCAAGCCGAGGCATCCTTA (SEQ ID NO:366) and reverse primer: TCCAAAGCGTGTTCCCTTCGGTAT (SEQ ID NO:367)). 1 μL of cDNA from the aRNA protocol (before any amplification) is used as the template with Platinum Taq DNA Polymerase using the recommended protocol (Invitrogen, Carlsbad, Calif.). If plasma cell contamination was detected in a sample, the sample was discarded.

Preparation of the target polynucleotides from B cells. RNA was isolated from the sorted B cells using TRIZOL (Invitrogen, Carlsbad, Calif.). Samples containing 100 B cells do not contain sufficient RNA for direct analysis in a microarray. Therefore, an established antisense RNA (aRNA) amplification protocol designed to minimize introduction of bias was used (MEGAscript T7 Kit, Ambion, Austin, Tex.). Two rounds of amplification provided sufficient RNA for hybridization. Amide-modified UTP was incorporated in the second round product and was used for fluorescent labeling of the samples. The RNA samples were labeled using ULYSIS dyes according to the manufacturer's instructions (Invitrogen-Molecular Probes, Eugene Oreg.).

Hybridization of the target polynucleotides to the microarray and scanning. Labeled aRNA samples were mixed with 1 μg of poly-A RNA as a blocking reagent and hybridization buffer (50% formamide, 5×SSC, 0.1% SDS and 0.1 mg/mL BSA) and added to the microarray slide. Hybridizations were performed in a 45° C. water bath overnight. After hybridization, microarrays were washed in 2×SSC, 0.1% SDS at 42° C. for 5 minutes, two times in 1×SSC at room temperature, two times in 0.1×SSC, and water for 30 seconds. Slides were then dried by centrifugation at 2,500 RPMs and immediately scanned using Axon GenePix 4000B (Molecular Devices, Sunnyvale, Calif.). Data analysis was performed on the scanned image using commercially available software and software designed in our lab. (GeneSpring, Agilent, Palo Alto).

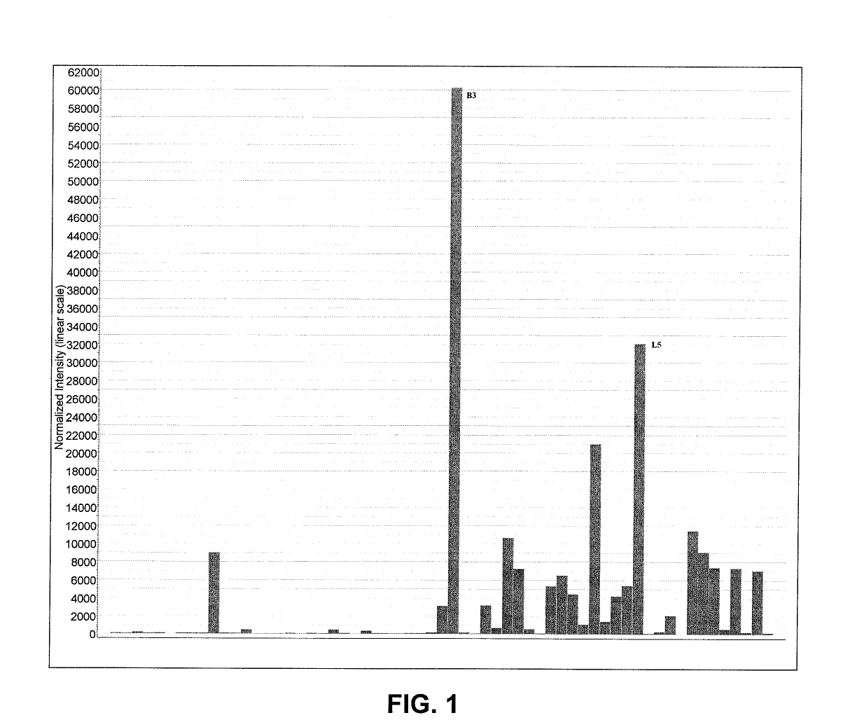

Specificity of the microarray. To establish that the selected oligonucleotide sequences (represented in Table 2) were specific for the indicated light chain variable regions, RNA prepared from human light chain variable gene clones was used in the array. The B cell clones were obtained through a Material Transfer Agreement with the Mayo Clinic (Rochester, Minn.) and each of the light chain variable regions is known. FIG. 1 is a representative example of microarray data from a single B cell clone known to express the B3 light chain. This experiment allows for assessment of the level of cross-hybridization of the oligonucleotides in the microarray and provides an example of how this method can be used to characterize the light chain in plasma cell diseases. Similar experiments have been performed using the L12 light chain clone and are planned for the O8/18, V1-19, V1-16 and V1-22 light chains. Specificity of the mouse light chain oligonucleotides has been determined using RNA from hybridomas.

Use of Reference Sequence in Light Chain Microarray. A reference sequence is used to control for differences in probe hybridization efficiency, spotting inconsistencies and print batch differences and other variations that may influence spot intensity. The reference sequence is composed of equal-molar concentrations of DNA oligonucleotides complementary to the light chain probes. A large amount of the reference sequence has been synthesized and stored. It could also be re-synthesized if necessary. The reference sample is labeled with one fluorophore and the sample nucleic acid is labeled with a second fluorophore. Thus, spots or probe hybridization efficiency will be reflected in the intensity reading of the reference sample (a spot/probe with low hybridization efficiency will have a low intensity, while a spot/probe with high hybridization efficiency will have a high intensity). Thus, the sample of interest can be normalized on a probe-by-probe (gene-by-gene) basis according to the reference sample intensity of a particular probe.

Cross-hybridization Quantification and Incorporation into Data Analysis. The relatedness of the light chain V genes is reflected in the germline sequence similarity. In some cases, V genes have been duplicated and have not diverged (for example, O2 and O12 are identical, as are others). Other V genes have diverged slightly and share significant sequence similarity. While the oligonucleotide species described above were designed to exploit all possible differences, some of them are very similar to V genes other than the gene they were designed to interrogate. Thus, cross-hybridization between an oligonucleotide species and a related V gene is a concern. One example of this cross-hybridization is demonstrated in FIG. 1 where the B3 nucleic acid hybridizes to other oligonucleotide species, including the oligonucleotide species designed to hybridize to L5. We are currently in the process of testing each V gene sequence individually to determine the extent of cross-hybridization with all of the oligonucleotides species (we have completed over 60% of these hybridizations). When cloned V gene sequences were not available, the complementary sequence to that probe was synthesized, labeled and hybridized to the array. The data from these hybridizations is being compiled in a matrix. This matrix of cross-hybridization will then contain all of the information necessary to distinguish a real signal from cross-hybridization. This will be done by crossing the data generated by a sample with the inverse of this matrix. The output of this computation is the true signal.

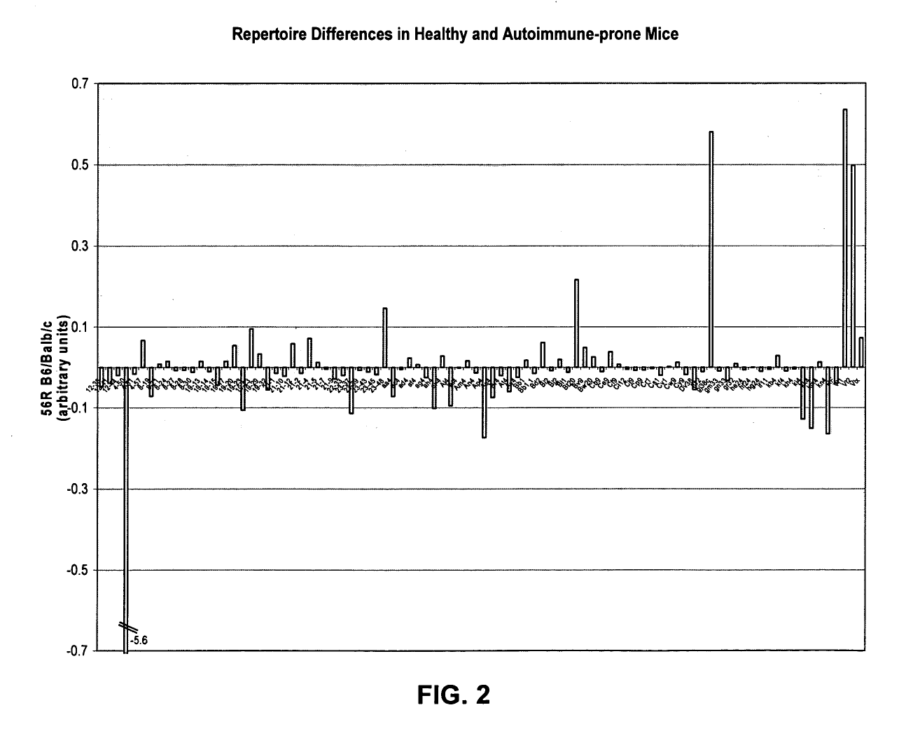

Repertoire differences in autoimmune-prone and non-autoimmune prone mice. C57/B6 mice with the 56R heavy-chain transgene develop auto-antibodies at a very young age, while Balb/c mice (without any transgene) remain healthy and do not develop auto-antibodies. See Sekiguchi et al., J. Immunol. 176:6879-6887 (2006). The repertoire from two 56R transgenic C57/B6 mice with detectable auto-antibodies was compared with six Balb/c mice without any evidence of autoimrnunity using the light chain variable region microarray and the results are depicted in FIG. 2.

Briefly, B cells were sorted for each mouse independently, RNA was prepared and hybridization with the microarray performed as described in detail above. After hybridization, image analysis was completed using Axon GenePix and the median intensity for all replicate spots averaged. Intensity levels across samples were normalized by comparison with kappa-constant values from the same sample. These normalized values were then averaged for the 56R transgenic C57/B6 mice and the Balb/c mice. The Balb/c normalized and averaged values were then subtracted from the 56R transgenic C57/B6 normalized and averaged values for each gene. Thus, a positive value in FIG. 2 corresponds to a light chain being overrepresented in a 56R transgenic C57/B6 autoimmune mouse, while a negative value corresponds to overrepresentation of a light chain variable region in Balb/c mice. The following L-chain V gene spots had values below background and were not included in the analysis: 12-46, 21-3, 8-16, 8-34, ae4 and ba9.

As shown in FIG. 2, several of the light chain variable genes were overrepresented in the autoimmune prone transgenic mice, namely 23-48, Bt20, gj38c, V11, and V12. Several of these light chain variable genes, including Bt20 and gj38c have been linked to autoimmune disease in this mouse model using traditional B cell cloning and sequencing or PCR-based analysis of the light chain variable gene. See Li et al., Immunity 15:947:957 (2001) and Table 1.

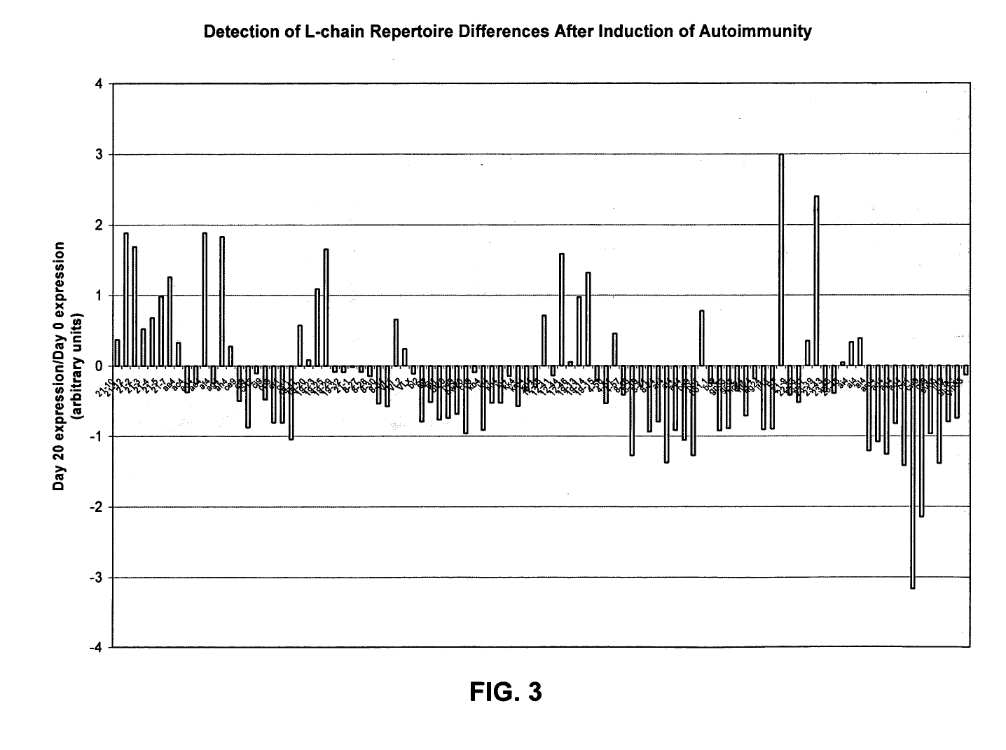

L-chain repertoire changes with induced autoimmunity. Chronic graft-versus-host (cGvH) disease was induced by injection of allogenic CD4+ T cells from a bm12 mouse into a 56R heavy-chain transgenic B6 mouse as previously described. See Sekiguchi et. al., Proc. Natl. Acad. Sci. U.S.A. 84:9150-9154 (2003) which is incorporated herein by reference in its entirety. The B cell light chain repertoire was sampled 20 days post-induction using the light chain variable gene microarray as described above and the results are presented in FIG. 3. Anti-DNA antibodies were increased at day 20 post-induction in this mouse compared with day 0 and were higher than a littermate control 56R transgenic mouse that did not undergo cGvH induction as measured by ELISA.

Briefly, RNA was prepared from 100 B cells from a cGvH-induced 56R transgenic mouse and a control 56R transgenic mouse. The RNA was labeled and hybridized to the array. The microarray image was analyzed using the Axon GenePix, and the median intensity for all replicate spots was averaged for each sample. Intensity levels were normalized by comparing the average intensity of each light chain variable gene with the kappa-constant gene intensity for the same sample. These values from the control (no cGvH) 56R mouse were then subtracted from the day 20 cGvH 56R values for each gene and plotted on the y-axis. Positive values correspond to an expansion of light chain variable genes after induced autoimmunity, and negative values correspond to light chain variable genes that are underrepresented after induction of autoimmunity.

As shown in FIG. 3, several of the light chain variable genes were overrepresented in the autoimmune mice at day 20 post-induction, while other light chain variable genes were underrepresented in these animals as compared to untreated control transgenic mice. The light chain variable genes overrepresented and underrepresented in this model were distinct from those identified as overrepresented in 56R as compared to Balb/c mice in FIG. 2 and are distinct from the previously reported light chain variable genes linked to autoimmunity Sekiguchi et al., J. Immunol. 176:6879-6887 (2006), Table 1, and unpublished data.

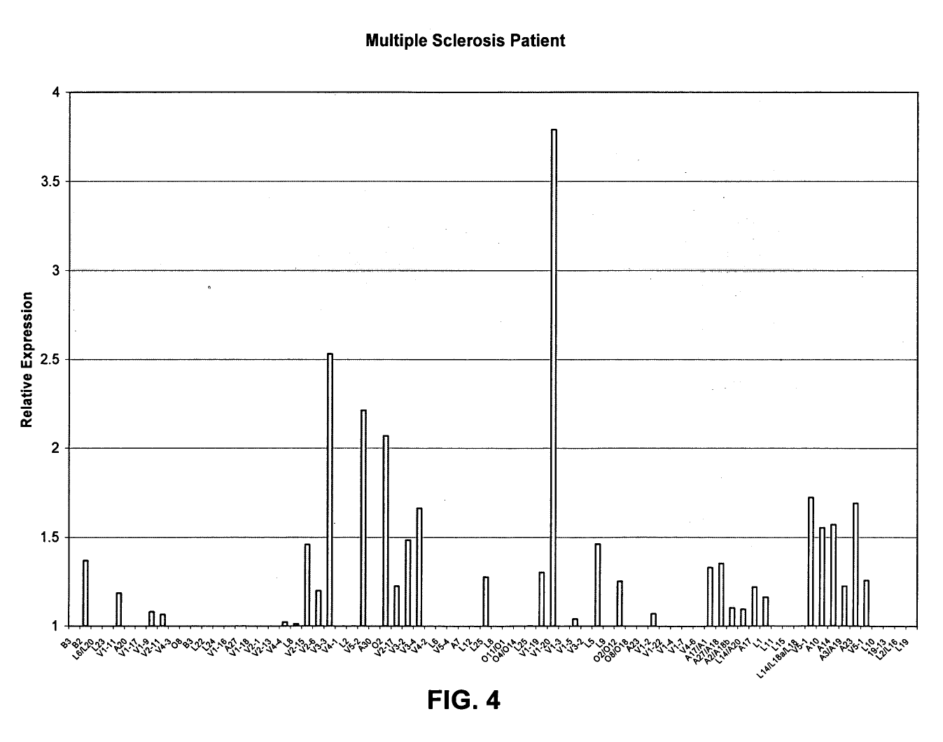

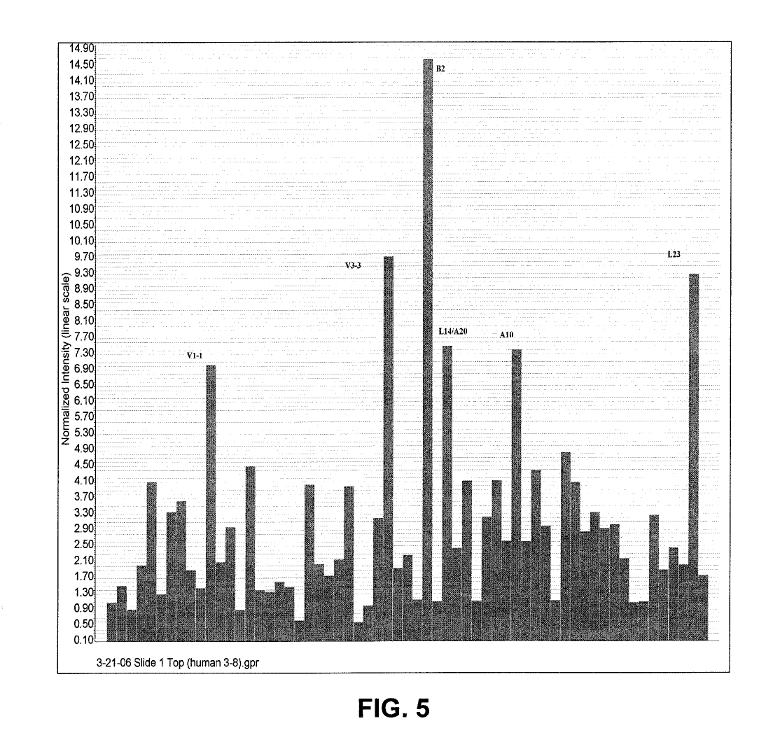

Light Chain Variable Detection in Human Autoimmune Disease. Reports in the literature suggest multiple sclerosis (MS) patients display a restricted cerebral-spinal fluid (CSF) B cell repertoire. See Monson et al., J. Neuroimmunol. 158:170-181 (2005) and Colombo et al., J. Immunol. 164:2782-2789 (2000) which are incorporated herein by reference in their entireties. Therefore, this disease was chosen to test the microarray and determine if the light chain variable regions identified in Table 1 were found in MS patients. B cells were harvested from the CSF of an untreated MS patient and from three individuals who do not have MS. The cells were sorted, the RNA isolated, amplified, labeled and hybridized to the microarray as described above. FIG. 4 depicts the light chain variable gene expression from the MS patient normalized to the kappa constant gene after averaging the replicates. The data demonstrate that a subset of light chain variable genes is expressed in the MS patient. Notably, several of the expressed genes correlate to the light chains hypothesized to be important in autoimmune pathology listed in Table 1, namely B2, O8/O18, L25 and V2-15. FIG. 5 shows the light chain variable gene expression as fluorescence intensity normalized to the kappa constant region as a ratio to the light chain variable gene expression in three healthy individuals. The light chain variable regions that were differentially expressed are noted in the Figure. As depicted in FIG. 5, the B2 light chain was overrepresented in the MS patient as compared to the healthy individuals and this chain has structural properties similar to pathogenic light chains in the mouse as indicated in Table 1.

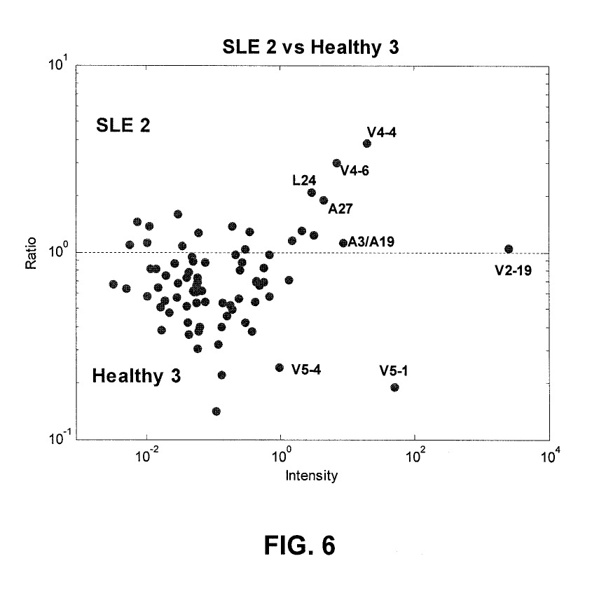

Light Chain Repertoire Differences in SLE. This method has detected V gene light chain repertoire differences between an individual with a clinical diagnosis of SLE and a healthy individual with no know autoimmunity. In this example, peripheral blood was isolated from these two individuals. B cells of the CD20⁺CD138⁻CD27⁻CD38⁻ phenotype were sorted and prepared as described above. Each sample was labeled with Alexa 647 dyes and mixed with a reference sequence labeled with Alexa 555 (Invitrogen-Molecular Probes, Eugene Oreg.). The samples were independently hybridized, washed and scanned. Comparisons were made by performing global intensity normalization for each fluorescent channel on each array. These were used to generate a ratio of sample:reference, and this sample:reference ratio was compared between arrays to generate FIG. 6. As can be seen from this figure, some V genes are overrepresented in this SLE patient compared with this healthy individual (the points above the line such as V4-4, V4-6, L24 and A27). Additionally, some V genes are underrepresented in this SLE patient such as V5-4 and V5-1.

Various features of the invention are set forth in the following claims.

We claim:

1. A microarray comprising a plurality of oligonucleotide species, each species capable of hybridizing to a polynucleotide comprising a sequence or a complement thereof, the sequence encoding at least a portion of a light chain variable region, and wherein each of the plurality of oligonucleotide species is at least 20 nucleotides long.

2. The microarray of claim 1, wherein the light chain variable region is a vertebrate light chain variable region.

3. The microarray of claim 1, wherein the light chain variable region is a human light chain variable region.

4. The microarray of claim 1, wherein each of the plurality of oligonucleotide species is at least 40 nucleotides long.

5. The microarray of claim 1, wherein each of the plurality of oligonucleotide species is at least 60 nucleotides long.

6. The microarray of claim 1, wherein the plurality of oligonucleotide species comprises at least two oligonucleotide species substantially similar to the oligonucleotides of Table 2, Table 3, Table 4, or Table 5, or complements of the oligonucleotides of Table 2, Table 3, Table 4, or Table 5.

7. The microarray of claim 1, wherein the plurality of oligonucleotide species comprise at least 20 of the oligonucleotides of Table 2, Table 3, Table 4, or Table 5, or complements of the oligonucleotides of Table 2, Table 3, Table 4, or Table 5.

8. The microarray of claim 1, wherein each oligonucleotide species is immobilized at a distinct address on a substrate.

9. The microarray of claim 1, wherein at least one of the light chain variable regions is associated with a disease.

10. The microarray of claim 9, wherein the at least one light chain variable region is associated with a systemic autoimmune disease.

11. The microarray of claim 1, wherein the plurality of oligonucleotide species comprises an oligonucleotide comprising a sequence encoding at least a portion of a light chain variable region of mBT20, mBW20, mGJ38C, mVLX, m21-4, m12-38, m12-46, O8, O18, L25, B2, L11, L22, L10, V2-8, V2-14, V2-15, V2-19, A5, or complements of mBT20, mBW20, mGJ38C, mVLX, m21-4, m12-38, m12-46, O8, O18, L25, B2, L11, L22, L10, V2-8, V2-14, V2-15, V2-19, or A5.

12. A method of characterizing the light chain variable gene expression in a subject comprising:
a) isolating B cells from the subject;
b) preparing target polynucleotides from the B cells;
c) hybridizing the target polynucleotides to a microarray comprising a plurality of oligonucleotide species at least 20 nucleotides long, each species capable of hybridizing to at least one of the target polynucleotides comprising a sequence or a complement thereof, the sequence encoding at least a portion of a light chain variable region; and
d) detecting the hybridization.

13. A method of identifying light chain variable genes associated with a disease, comprising comparing the light chain variable gene expression in a first subject with the disease to the light chain variable gene expression in a second subject that does not have the disease, a difference in light chain variable gene expression between the first and second subjects indicating that expression of the light chain variable gene is associated with the disease.

14. The method of claim 13, wherein the disease is a systemic autoimmune disease.

15. The method of claim 14, wherein the systemic autoimmune disease is selected from the group consisting of systemic lupus erythematosus, multiple sclerosis, rheumatoid arthritis, scleroderma, Sjogren's syndrome, mixed connective tissue disease, amyloidosis, and psoriasis.

16. The method of claim 13, wherein the disease is cancer.

17. The method of claim 16, wherein the cancer is a B cell cancer.

18. The method of claim 13, wherein the disease is an immunodeficiency disease.

19. A method of monitoring a disease state in a subject comprising comparing expression in the subject of a light chain variable gene associated with the disease at two or more different time points.

20. A method of a evaluating the effect of a therapy or therapeutic agent on expression of a light chain variable gene associated with a disease in a subject, comprising comparing expression of the light chain variable gene expression in the subject before and after treatment.

21. A kit comprising the microarray of claim 1.
